SEQUENCE LISTING (23 sequences)

SEQ ID NO: 1 - protein, 197 aa, Homo sapiens
MTLLPGLLFLTWLHTCLAHHDPSLRGHPHSHGTPHCYSAEELPLGQAPPHLLARGAKWGQALPVALVSSLEAASHRGRHERPSATTQCPVLRPEEVLEADTHQRSISPWRYRVDTDEDRYPQKLAFAECLCRGCIDARTGRETAALNSVRLLQSLLVLRRRPCSRDGSGLPTPGAFAFHTEFIHVPVGCTCVLPRSV

SEQ ID NO: 2 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 3 - protein, 667 aa, Homo sapiens
MGSSRLAALLLPLLLIVIDLSDSAGIGFRHLPHWNTRCPLASHTDDSFTGSSAYIPCRTWWALFSTKPWCVRVWHCSRCLCQHLLSGGSGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRHLGLLILALLALLTLLGVVLALTCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 4 - protein, 194 aa, Mus musculus
MSLLLLGWLPTGMTHQDPPSWGKPRSHRTLRCYSAEELSHGQAPPHLLTRSARWEQALPVALVASLEATGHRRQHEGPLAGTQCPVLRPEEVLEADTHERSISPWRYRIDTDENRYPQKLAVAECLCRGCINAKTGRETAALNSVQLLQSLLVLRRQPCSRDGTADPTPGSFAFHTEFIRVPVGCTCVLPRSTQ

SEQ ID NO: 5 - protein, 197 aa, Macaca fascicularis
MTLLPGLLFLTWLHACLAHQDPFLRGHPHTHGTPRCYSAEELPLGQAPPHLLARGAKWGQALPVALVSSLEAAGHRRRHDRPSAATQCPVLRPEEVLEADTHQRSISPWRYRVDTDEDRYPQKLAFAECLCRGCIDPRTGRETAALNSVRLLQSLLVLRRRPCSRDGSGLPTPGAFAFHTEFIRVPVGCTCVLPRSV

SEQ ID NO: 6 - protein, 637 aa, Mus musculus
MGSPRLAALLLSLPLLLIGLAVSARVACPCLRSWTSHCLLAYRVDKRFAGLQWGWFPLLVRKSKSPPKFEDYWRHRTPASFQRKLLGSPSLSEESHRISIPSSAISHRGQRTKRAQPSAAEGREHLPEAGSQKCGGPEFSFDLLPEVQAVRVTIPAGPKASVRLCYQWALECEDLSSPFDTQKIVSGGHTVDLPYEFLLPCMCIEASYLQEDTVRRKKCPFQSWPEAYGSDFWQSIRFTDYSQHNQMVMALTLRCPLKLEASLCWRQDPLTPCETLPNATAQESEGWYILENVDLHPQLCFKFSFENSSHVECPHQSGSLPSWTVSMDTQAQQLTLHFSSRTYATFSAAWSDPGLGPDTPMPPVYSISQTQGSVPVTLDLIIPFLRQENCILVWRSDVHFAWKHVLCPDVSHRHLGLLILALLALTALVGVVLVLLGRRLLPGSGRTRPVLLLHAADSEAQRRLVGALAELLRTALGGGRDVIVDLWEGTHVARIGPLPWLWAARERVAREQGTVLLLWNCAGPSTACSGDPQAASLRTLLCAAPRPLLLAYFSRLCAKGDIPRPLRALPRYRLLRDLPRLLRALDAQPATLASSWSHLGAKRCLKNRLEQCHLLELEAAKDDYQGSTNSPCGFSCL

SEQ ID NO: 7 - protein, 5 aa, Artificial Sequence
  source: /note=“Description of Artificial Sequence Synthetic peptide”
DYAMH

SEQ ID NO: 8 - protein, 17 aa, Artificial Sequence
  source: /note=“Description of Artificial Sequence Synthetic peptide”
YIGGVGEGTQYAESVKG

SEQ ID NO: 9 - protein, 11 aa, Artificial Sequence
  source: /note=“Description of Artificial Sequence Synthetic peptide”
GFAIRYYGFDY

SEQ ID NO: 10 - protein, 7 aa, Artificial Sequence
  source: /note=“Description of Artificial Sequence Synthetic peptide”
GFTVSDY

SEQ ID NO: 11 - protein, 6 aa, Artificial Sequence
  source: /note=“Description of Artificial Sequence Synthetic peptide”
GGVGEG

SEQ ID NO: 12 - protein, 11 aa, Artificial Sequence
  source: /note=“Description of Artificial Sequence Synthetic peptide”
GFAIRYYGFDY

SEQ ID NO: 13 - protein, 11 aa, Artificial Sequence
  source: /note=“Description of Artificial Sequence Synthetic peptide”
SGDKLGDKYAY

SEQ ID NO: 14 - protein, 7 aa, Artificial Sequence
  source: /note=“Description of Artificial Sequence Synthetic peptide”
QDSKRPS

SEQ ID NO: 15 - protein, 10 aa, Artificial Sequence
  source: /note=“Description of Artificial Sequence Synthetic peptide”
QVFTFPLVTT

SEQ ID NO: 16 - protein, 120 aa, Artificial Sequence
  source: /note=“Description of Artificial Sequence Synthetic polypeptide”
EVQLLESGGGLVQPGGSLRLSCAASGFTVSDYAMHWVRQAPGKGLEWVSYIGGVGEGTQYAESVKGRFTISRDNSKNTLYLQMNSLRAEDTAVYYCARGFAIRYYGFDYWGQGTLVTVSS

SEQ ID NO: 17 - protein, 110 aa, Artificial Sequence
  source: /note=“Description of Artificial Sequence Synthetic polypeptide”
SYELTQPPSVSVSPGQTASITCSGDKLGDKYAYWYQQKPGQSPVLVIYQDSKRPSGIPERFSGSNSGNTATLTISGTQAEDEADYYCQVFTFPLVTTVFGGGTKLTVLGQ

SEQ ID NO: 18 - protein, 450 aa, Artificial Sequence
  source: /note=“Description of Artificial Sequence Synthetic polypeptide”
EVQLLESGGGLVQPGGSLRLSCAASGFTVSDYAMHWVRQAPGKGLEWVSYIGGVGEGTQYAESVKGRFTISRDNSKNTLYLQMNSLRAEDTAVYYCARGFAIRYYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 214 aa, Artificial Sequence
  source: /note=“Description of Artificial Sequence Synthetic polypeptide”
SYELTQPPSVSVSPGQTASITCSGDKLGDKYAYWYQQKPGQSPVLVIYQDSKRPSGIPERFSGSNSGNTATLTISGTQAEDEADYYCQVFTFPLVTTVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 20 - DNA, 330 bp, Artificial Sequence
  source: /note=“Description of Artificial Sequence Synthetic polynucleotide”
tcctacgagctgacccagcccccctccgtgtccgtgtctcctggccagaccgcctccatcacctgttccggcgacaagctgggcgataagtacgcctactggtatcagcagaagcccggccagtcccccgtgctggtcatctaccaggactccaagcggccctccggcatccctgagcggttctccggctccaactccggcaacaccgccaccctgaccatctccggcacccaggccgaggacgaggccgactactactgccaggtgttcaccttccccctggtcaccaccgtgttcggcggaggcaccaagctgaccgtgctgggccag

SEQ ID NO: 21 - DNA, 360 bp, Artificial Sequence
  source: /note=“Description of Artificial Sequence Synthetic polynucleotide”
gaggtgcagctgctggaatccggcggaggactggtgcagcctggcggctccctgagactgtcttgcgccgcctccggcttcaccgtgtccgactacgctatgcactgggtccgacaggcccctggcaagggcctggaatgggtgtcctatatcggcggcgtgggcgagggcacccagtacgctgagtctgtgaagggccggttcaccatctcccgggacaactccaagaacaccctgtacctgcagatgaactccctgcgggccgaggacaccgccgtgtactactgtgccagaggcttcgccatccggtactacggcttcgactactggggccagggcaccctggtcaccgtgtctagc

SEQ ID NO: 22 - DNA, 642 bp, Artificial Sequence
  source: /note=“Description of Artificial Sequence Synthetic polynucleotide”
tcctacgagctgacccagcccccctccgtgtccgtgtctcctggccagaccgcctccatcacctgttccggcgacaagctgggcgataagtacgcctactggtatcagcagaagcccggccagtcccccgtgctggtcatctaccaggactccaagcggccctccggcatccctgagcggttctccggctccaactccggcaacaccgccaccctgaccatctccggcacccaggccgaggacgaggccgactactactgccaggtgttcaccttccccctggtcaccaccgtgttcggcggaggcaccaagctgaccgtgctgggccagcctaaggccgctccctccgtgaccctgttccccccatcctccgaggaactgcaggccaacaaggccaccctggtctgcctgatctccgacttctaccctggcgccgtgaccgtggcctggaaggccgacagctctcctgtgaaggccggcgtggaaaccaccaccccctccaagcagtccaacaacaaatacgccgcctcctcctacctgtccctgacccccgagcagtggaagtcccaccggtcctacagctgccaggtcacacacgagggctccaccgtggaaaagaccgtggcccctaccgagtgctcc

SEQ ID NO: 23 - DNA, 1350 bp, Artificial Sequence
  source: /note=“Description of Artificial Sequence Synthetic polynucleotide”
gaggtgcagctgctggaatccggcggaggactggtgcagcctggcggctccctgagactgtcttgcgccgcctccggcttcaccgtgtccgactacgctatgcactgggtccgacaggcccctggcaagggcctggaatgggtgtcctatatcggcggcgtgggcgagggcacccagtacgctgagtctgtgaagggccggttcaccatctcccgggacaactccaagaacaccctgtacctgcagatgaactccctgcgggccgaggacaccgccgtgtactactgtgccagaggcttcgccatccggtactacggcttcgactactggggccagggcaccctggtcaccgtgtctagcgcctccaccaagggcccctccgtgttccctctggccccctccagcaagtccacctctggcggcaccgctgccctgggctgcctggtcaaggactacttccccgagcccgtgaccgtgtcctggaactctggcgccctgacctccggcgtgcacaccttccctgccgtgctgcagtcctccggcctgtactccctgtcctccgtcgtgaccgtgccctccagctctctgggcacccagacctacatctgcaacgtgaaccacaagccctccaacaccaaggtggacaagcgggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtgtccaacaaggccctgcctgcccccatcgaaaagaccatctccaaggccaagggccagccccgcgagccccaggtgtacacactgccccctagccgggaagagatgaccaagaaccaggtgtccctgacctgtctggtcaagggcttctacccctccgacattgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

(19) United States
(12) Patent Application Publication
(10) Pub. No.: US 2021/0253692 A1
(43) Pub. Date: Aug. 19, 2021

(54) High Concentration Liquid Antibody Formulations

(71) Applicants: GALAPAGOS NV, Mechelen (BE); MORPHOSYS AG, Planegg (DE)

(72) Inventors: Martin DOMNOWSKI, Munich (DE); Roy EYLENSTEIN, Ilmnitz (DE); Jan JAEHRLING, Neuried (DE); Daniel WEINFURTNER, Seeshaupt (DE)

(21) Appl. No.: 17/256,701

(22) PCT Filed: Jul. 2, 2019
(86) PCT No.: PCT/IB2019/055635

§ 371 Date: Dec. 29, 2020

(30) Foreign Application Priority Data
Jul. 3, 2018 (EP) 18181380.9

(51) Int. Cl. C07K 16/24; A61K 47/26; A61K 47/18

(52) U.S. Cl. CPC C07K 16/244; A61K 2039/54; A61K 47/183; A61K 47/26

(57) ABSTRACT

The present disclosure describes high concentration liquid formulations of a pharmaceutically active antigen binding protein, for example a monoclonal antibody. Such formulations comprise, in addition to the antigen binding protein, at least 80 mM of a buffering agent and at least 80 mM of a stabilizer. In addition, the present disclosure is related to pharmaceutical formulations of an anti-IL-17C antibody and provides methods of making and methods of using such formulations.

### FIELD OF THE DISCLOSURE

The present disclosure relates to high concentration liquid formulations of a pharmaceutically active antigen binding protein, for example a monoclonal antibody. Such formulations comprise, in addition to the antigen binding protein, at least 80 mM of a buffering agent and at least 80 mM of a stabilizer. In addition, the present disclosure is related to pharmaceutical formulations of an anti-IL-17C antibody and provides methods of making and methods of using such formulations.

### BACKGROUND

The pharmaceutical use of antibodies has increased over the past years. In many instances such antibodies are injected via the intravenous (IV) route. Alternative administration pathways are subcutaneous or intramuscular injection, which offer potential advantages in terms of patient compliance and ease of administration. However these injection pathways require high protein concentration in the final solution to be injected.

Accordingly, there is a desire to provide highly concentrated, stable pharmaceutical formulations of therapeutically active antigen binding proteins such as antibodies for subcutaneous injection. The advantage of subcutaneous injections is that it allows the medical practitioner to perform it in a rather short intervention with the patient. Moreover the patient can be trained to perform the subcutaneous injection by himself. Such self-administration is particularly useful during maintenance dosing because no hospital care is needed (reduced medical resource utilization).

However, due to the very limited volume for subcutaneous injection, these protein concentrations will reach more than 100 mg/mL, which reveals several challenges regarding protein stabilities, protein-protein interactions and increasing viscosity. Viscosity is not only an issue regarding the biophysical and biochemical properties of the therapeutic protein, but also for the delivery and manufacturing of such highly concentrated protein solutions. The higher the viscosity of the solution the longer it takes to inject such a viscous solution via syringe and needle. So the aspect of syringability is influenced by the viscosity and needs to be considered during the development of a high-concentration liquid formulation. Most commercially available auto-injectors are limited to solution viscosities of lower than 20 cP. Therefore, viscosity is a very crucial factor for the development of a high-concentration liquid formulation of therapeutic antibodies regarding manufacturing and the respective delivery of the product.

To address these problems the development of suitable formulation compositions is of utmost importance.

However, specific antibodies have properties, like undesired self-interaction, which makes them less suitable for a high concentration formulation. This self-interaction propensity may lead to oligomerization, aggregation and high viscosities at high protein concentrations. Several assays like self-interaction chromatography or the determination of the diffusion interaction parameter, which detect and quantify antibody self-interaction are available. For example, Publicover and Vincze (U.S. Pat. No. 7,514,938B2) describe the use of dielectric relaxation spectroscopy (DRS) to probe the interaction and aggregation of micron and sub- micron scale particles coated with protein, including antibodies. Holman et al, (US20070291265A1) describe a bifurcated fiber optic system for measuring light scattering and concentration signals to measure aggregation of macromolecules. Obrezanova et al. (mAbs, 7(2): 352-363, 2015) describe the use of size exclusion high pressure liquid chromatography (SE-HPLC) and an oligomer detection assay, which is an optical density microtiter plate antibody capture assay, to systematically measure the aggregation propensity of over 500 antibodies. Geoghegan et al. (mAbs, 8(5): 941-950, 2016) describe the use of hydrophobic interaction chromatography (HIC) retention time, affinity-capture SINS, and dynamic light scattering to measure monoclonal antibody self-interaction, viscosity and stability. An overview of the current methods used to assess colloidal protein interactions is provided by Geng, et al. (J Pharm Sci., 103(11): 3356-3363, 2014).

Therefore, a need exists to develop high concentration formulations for antibodies having a self-interaction propensity.

### SUMMARY OF THE DISCLOSURE

The present disclosure provides a pharmaceutical formulation for an antigen binding protein comprising that antigen binding protein, a buffering agent and a stabilizer. More particularly, the present disclosure provides a pharmaceutical formulation for an antigen binding protein comprising about 100 to 250 mg/mL antigen binding protein, about 80 to about 120 mM of a buffering agent providing a pH of about 5.0 to about 7.0 and about 80 to about 120 mM of a stabilizer. In another aspect the antigen binding protein has a self-interaction propensity.

In one aspect, the present disclosure provides a pharmaceutical formulation for an antigen binding protein comprising about 100 to 250 mg/mL, 125 to 200 mg/mL, 130 to 180 mg/mL, 140±10 mg/mL, 150±10 mg/mL, 160±10 mg/mL, 170±10 mg/mL, 140 mg/mL, 150 mg/mL, 160 mg/mL, 170 mg/mL antigen binding protein.

In another aspect, the present disclosure provides a pharmaceutical formulation for an antigen binding protein comprising about 100 to 250 mg/mL antigen binding protein; about 80 to 120 mM of a buffering agent and about 80 to 120 mM of a stabilizer. In another aspect the concentration of the buffer agent is about 90 to 110 mM and the concentration of the stabilizer is about 90 to 110 mM.

In another aspect, the present disclosure provides a pharmaceutical formulation for an antigen binding protein comprising about 100 to 250 mg/mL antigen binding protein; about 80 to 120 mM of a histidine buffer and about 80 to 120 mM of a stabilizer. In another aspect the histidine buffer concentration is about 100 mM. In a further aspect the histidine buffer concentration is 100 mM. In another aspect the histidine buffer is histidine hydrochloride. In another aspect the pharmaceutical formulation has a pH of about 5.0 to about 7.0. In another aspect the pharmaceutical formulation has a pH of 6.0. In another aspect the antigen binding protein has a self-interaction propensity.

In one aspect the present disclosure provides a pharmaceutical formulation for an antigen binding protein comprising that antigen binding protein, a buffering agent and a stabilizer. In another aspect the stabilizer is an amino acid. In a further aspect the amino acid is arginine.

In one aspect the present disclosure provides a pharmaceutical formulation for an antigen binding protein comprising that antigen binding protein, a buffering agent and arginine. In another aspect the arginine concentration is about 100 mM. In a further aspect the arginine concentration is 100 mM.

In one aspect the present disclosure provides a pharmaceutical formulation for an antigen binding protein comprising that antigen binding protein a buffering agent a stabilizer and further comprising about 0.005 to 0.05% (w/v) of a nonionic surfactant. In another aspect the nonionic surfactant is polysorbate 20 or polysorbate 80. In a further aspect the polysorbate 20 or polysorbate 80 concentration is about 0.02% (w/v).

In one aspect the present disclosure provides a pharmaceutical formulation for an antigen binding protein for subcutaneous or intramuscular administration. In another aspect the present disclosure provides a pharmaceutical formulation for an antigen binding protein in liquid form, reconstituted form, lyophilized or spray dried form.

In another aspect the antigen binding protein has a self-interaction propensity.

In another aspect the antigen binding protein is a monoclonal antibody.

In one aspect the present disclosure provides a pharmaceutical formulation for an antigen binding protein, wherein the antigen binding protein is an antibody. In another aspect the antibody is a monoclonal antibody. In another aspect the monoclonal antibody has a propensity for self-interaction.

WO2017/140831 discloses an antibody that binds IL-17C and inhibits binding of IL-17C to its receptor throughout relevant species (e.g. human, mouse and cynomolgus monkey). Such antibody proved to be effective in various in vivo mouse models for atopic dermatitis and psoriasis.

In one aspect the present disclosure provides a pharmaceutical formulation for an IL-17C antibody. In one aspect the IL-17C antibody binds to human IL-17C (SEQ ID NO: 1). In another aspect the IL-17C antibody comprises heavy and light chain variable regions comprising amino acid sequences that are 90% identical to SEQ ID NOs: 16 and 17 respectively. In another aspect the IL-17C antibody comprises heavy and light chain variable regions comprising amino acid sequences according to SEQ ID NOs: 16 and 17 respectively.

In one aspect the present disclosure provides an injection device comprising a pharmaceutical formulation according to the present disclosure.

In another aspect the present disclosure provides for a method of treating a disease or condition which is amenable to treatment with an IL-17C antibody in a subject comprising administering a formulation according the present disclosure in a subject in an amount effective to treat the disease or condition. In one aspect the disease or condition is an inflammatory disease or disorder.

In another aspect the present disclosure provides for a kit comprising one or more vials containing the formulation according to the present disclosure and instructions for subcutaneous administration of the formulation to a patient.

In another aspect the present disclosure provides for an injection device comprising a stable anti-IL-17C antibody formulation described herein.

In another aspect the present disclosure provides for a pharmaceutical formulation according to the present disclosure for therapeutic use, such as the treatment of inflammatory disorders like e.g. rheumatoid arthritis, psoriasis, pulmonary inflammation, COPD and/or the treatment of atopic dermatitis (AD), including moderate-to-severe AD.

### BRIEF DESCRIPTION OF THE DRAWINGS

FIG. 1: Overview of the experimental procedure for the BLI-based self-interaction assay. (1) Baseline step (2) Loading step by using the mAb of interest ([antibody]=25 nM) (3) Quenching step by using human Fc fragment (1 μM) (4) association reaction ([antibody]=2 μM). The abscissa shows the time course of the assay.

FIG. 2: Overview of the 3-step DoE approach leading to the high protein concentration formulation of MAB#1

FIG. 3: Response contour plots of self-interaction propensity. As formulations, only histidine-HCl based buffers were included without surfactant.

FIG. 4: Response contour plots of monomer content (right). As formulations, only histidine-HCl based buffers were included without surfactant.

FIG. 5: Concentration procedure and SEC analysis of the evaluation study performed after the first screening round. Concentration was carried out in lab scale by centrifugation at 1500×g using CentriPrep Ultracel YM-30 (Merck Millipore).

FIG. 6: Response contour plots of self-interaction propensity and monomer content (right) of the second experimental design to optimize the histidine-hydrochloride based formulation.

FIG. 7: Response contour plots of monomer content of the second experimental design to optimize the histidine-hydrochloride based formulation.

FIG. 8: Response contour plot of the change in monomer content of the third experimental design to optimize the histidine-hydroclroide based formulation regarding surfactant and arginine concentration.

### DEFINITIONS AND DETAILED DESCRIPTION

It is to be understood that this disclosure is not limited to particular methods, reagents, compounds, compositions, or biological systems, which can, of course, vary. It. is also to be understood that the terminology used herein is for the purpose of describing particular embodiments only, and is not intended to be limiting.

As used in this specification and the appended claims, the singular forms “a”, “an”, and “the” include plural referents unless the content clearly dictates otherwise. Thus, for example, reference to “a polypeptide” includes a combination of two or more polypeptides, and the like.

“About” as used herein when referring to a measurable value such as an amount, a temporal duration, and the like, is meant to encompass variations of ±20% or ±10%, including ±5%, ±1%, and ±0.1% from the specified value, as such variations are appropriate to perform the disclosed methods. Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, the preferred materials and methods are described herein, in describing and claiming the present disclosure, the following terminology will be used.

The terms “pharmaceutical formulation” or “formulation” refer to a preparation which is in such form as to permit the biological activity of the active ingredient to be effective, and which contains no additional components which are unacceptably toxic to a subject to which the formulation would be administered. Such formulations are sterile. A “sterile” formulation is aseptic or free from all living microorganisms and their spores.

The term “viscosity” refers to the internal resistance to flow exhibited by a fluid at a specified temperature; the ratio of shearing stress to rate of shear. A liquid has a viscosity of one poise if a force of 1 dyne/square centimeter causes two parallel liquid surfaces one square centimeter in area and one square centimeter apart to move past one another at a velocity of 1 cm/second. One poise equals one hundred centipoise. In one embodiment, the viscosity of the formulation comprising buffering agent and stabilizer is less than about 50 cP, less than about 45 cP, less than about 40 cP, less than about 35 cP, less than about 30 cP, less than about 25 cP, less than about 20 cP, less than about 15 cP, or less than about 10 cP.

When referring to apparent viscosity, it is understood that the value of viscosity is dependent on the conditions under which the measurement was taken, such as temperature, the rate of shear and the shear stress employed. The apparent viscosity is defined as the ratio of the shear stress to the rate of shear applied. There are a number of alternative methods for measuring apparent viscosity. For example, viscosity can be tested by a suitable cone and plate, parallel plate or other type of viscometer or rheometer.

A “histidine buffer” is a buffer comprising the amino acid histidine. Examples of histidine buffers include histidine hydrochloride, histidine acetate, histidine phosphate, and histidine sulfate.

By “isotonic” is meant that the formulation has essentially the same osmotic pressure as human blood, isotonic formulations will generally have an osmotic pressure from about 250 to 350 rnOsm. Isotonicity can be measured using a vapor pressure or freezing-point depression type osmometer.

In certain embodiments the pharmaceutical formulation according to the present disclosure comprises a stabilizer. Stabilizers, include, but are not limited to human serum albumin (hsa), bovine serum albumin (bsa), α-casein, globulins, a-lactalbumin, LDH, lysozyme, myoglobin, ovalbumin, and RNase A. Stabilizers also include amino acids and their metabolites, such as, glycine, alanine (a-alanine, β-alanine), arginine, betaine, leucine, lysine, glutamic acid, aspartic acid, proline, 4-hydroxyproline, sarcosine, γ-aminobutyric acid (GAB A), opines (alanopine, octopine, strombine), and trimethylamine N-oxide (TMAO). In one embodiment the stabilizer is an amino acid. In one embodiment the amino acid is arginine. In one embodiment the arginine concentration is about 80 to 120 mM. In one embodiment, the arginine concentration is about 100+/−20 mM.

In certain embodiments the pharmaceutical formulation according to the present disclosure comprises a nonionic surfactant. Nonionic surfactants, include, but are not limited to, polyoxyethylensorbitan fatty acid esters (such as polysorbate 20 and polysorbate 80), polyethylene-polypropylene copolymers, polyethylene-polypropylene glycols, polyox ethylene-stearates, polyoxyethylene alkyl ethers, e.g. polyoxyethylene monolauryl ether, alkylphenylpolyoxyethylene ethers (Triton-X), polyoxyethylene-polyoxypropylene copolymer (Poloxamer, Piuronic), sodium dodecyl sulphate (SDS). In one embodiment the nonionic surfactant is polysorbate 20 or polysorbate 80. In one embodiment the polysorbate 20 or polysorbate 80 concentration is about 0.005 to 0.04% (w/v). In one embodiment, the polysorbate 20 or polysorbate 80 concentration is about 0.02% (w/v). In one embodiment the nonionic surfactant is polysorbate 20.

In certain embodiments the pharmaceutical formulation according to the present disclosure further comprises a metal chelator. Metal chelators, include, but are not limited to EDTA and EGTA. In one embodiment the metal chelator is EDTA. In one embodiment the EDTA concentration is about 0.01 to about 0.02 mM. In one embodiment, the EDTA concentration is about 0.05 mM.

As used herein, the terms “self-interaction” and “self-association” are interchangeable and refer to the non-specific binding of a specific protein to one or more of the same identical proteins. The term non-specific, refers to association by weak forces. The self-association of two or more identical intact antibodies via electrostatic, Van der Waals or hydrophobic interactions to form dimers, trimers or higher order multimers that are reversible or irreversible are “non-specific”. In a preferred embodiment the self- association is reversible. An antigen binding protein, such as an antibody or antibody fragment, is considered to have a self-interaction propensity if the parameter kD (diffusion interaction parameter) has a value that is below 0 mL/g. In one embodiment the antigen binding protein having a self-interaction propensity has a kD (diffusion interaction parameter) of less than 0 mL/g, or less than −5 mL/g, or less than −10 mL/g, or less than −15 mL/g, or less than −20 mL/g. In one embodiment the antigen binding protein having a self-interaction propensity has a kD (diffusion interaction parameter) of about −23 mL/g. In one embodiment the antigen binding protein having a self-interaction propensity has a kD (diffusion interaction parameter) of less than 0 mL/g, or less than −5 mL/g, or less than −10 mL/g, or less than −15 mL/g, or less than −20 mL/g in phosphate buffered saline. In one embodiment the antigen binding protein having a self-interaction propensity has a kD (diffusion interaction parameter) of about −23 mL/g in phosphate buffered saline. Preferably, the kD (diffusion interaction parameter) is determined using the dynamic light scattering method as described in Connolly et al., 2012, Biophys. J. Vol. 103 or Menzen et al., 2014, J. Pharm. Sci., Vol. 103 in phosphate buffered saline.

In one embodiment, the antigen binding protein is a monoclonal antibody or fragment thereof. In one embodiment, the monoclonal antibody or fragment thereof is mouse, chimeric, humanized, or fully human. In one embodiment, the monoclonal antibody or fragment thereof binds to IL-17C.

In one aspect the pharmaceutical formulation of the present disclosure is stable upon freezing and thawing, A “stable” formulation is one in which all the protein therein essentially retain their physical stability and/or chemical stability and/or biological activity upon storage at the intended storage temperature, e.g. 2-8° C. It is desired that the formulation essentially retains its physical and chemical stability, as well as its biological activity upon storage. The storage period is generally selected based on the intended shelf-life of the formulation. Furthermore, the formulation should be stable following freezing (to, e.g., −70° C.) and thawing of the formulation, for example following 1,2 or 3 cycles of freezing and thawing. Various analytical techniques for measuring protein stability are available in the art and are reviewed in Peptide and Protein Drug Delivery', 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Stability can be evaluated qualitatively and/or quantitatively in a variety of different, ways, including evaluation of aggregate formation (for example using size exclusion chromatography, by measuring turbidity, and/or by visual inspection); by assessing charge heterogeneity using cation exchange chromatography or capillary zone electrophoresis; amino-terminal or earboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc.

In one embodiment, the pharmaceutical formulation of the present disclosure is suitable for subcutaneous or intramuscular administration.

The term “IL-17C” refers to a protein known as interleukin 17C (identified in HUGO Gene Nomenclature Committee (HGNC) by ID 5983 and in Mouse genome Informatics (MGI) database by ID 2446486). IL-17C is in some older publications referred to as CX2 or IL-21, however, it should not be confused with IL-21 cytokine, which is specifically expressed in activated CD4+ T cells, but not most of other tissues (Parrish-Novak et al (2000). Nature 408 (6808): 57-63). Human IL-21 is located on Chromosome 4 and is identified in HGNC database by ID 6005.

Human IL-17C is located on Chromosome 16 and has the amino acid sequence of (UniProt Q9P0M4):

(SEQ ID NO: 1)
MTLLPGLLFLTWLHTCLAHHDPSLRGHPHSHGTPHCYSAEELPLGQAPP
HLLARGAKWGQALPVALVSSLEAASHRGRHERPSATTQCPVLRPEEVLE
ADTHQRSISPWRYRVDTDEDRYPQKLAFAECLCRGCIDARTGRETAALN
SVRLLQSLLVLRRRPCSRDGSGLPTPGAFAFHTEFIHVPVGCTCVLPRS
V

Mouse IL-17C has the amino acid sequence of (UniProt Q8K4C5):

(SEQ ID NO: 4)
MSLLLLGWLPTGMTHQDPPSWGKPRSHRTLRCYSAEELSHGQAPPHLLT
RSARWEQALPVALVASLEATGHRRQHEGPLAGTQCPVLRPEEVLEADTH
ERSISPWRYRIDTDENRYPQKLAVAECLCRGCINAKTGRETAALNSVQL
LQSLLVLRRQPCSRDGTADPTPGSFAFHTEFIRVPVGCTCVLPRSTQ

Cynomolgus monkey IL-17C has the amino acid sequence of (XP_005592825.1):

(SEQ ID NO: 5)
MTLLPGLLFLTWLHACLAHQDPFLRGHPHTHGTPRCYSAEELPLGQAPP
HLLARGAKWGQALPVALVSSLEAAGHRRRHDRPSAATQCPVLRPEEVLE
ADTHQRSISPWRYRVDTDEDRYPQKLAFAECLCRGCIDPRTGRETAALN
SVRLLQSLLVLRRRPCSRDGSGLPTPGAFAFHTEFIRVPVGCTCVLPRS
V

The term “IL-17RA” refers to a protein known as interleukin 17 receptor A. Human IL-17RA has the amino acid sequence of (UniProt Q96F46):

(SEQ ID NO: 2)
MGAARSPPSAVPGPLLGLLLLLLGVLAPGGASLRLLDHRALVCSQPGLN
CTVKNSTCLDDSWIHPRNLTPSSPKDLQIQLHFAHTQQGDLFPVAHIEW
TLQTDASILYLEGAELSVLQLNTNERLCVRFEFLSKLRHHHRRWRFTFS
HFVVDPDQEYEVTVHHLPKPIPDGDPNHQSKNFLVPDCEHARMKVTTPC
MSSGSLWDPNITVETLEAHQLRVSFTLWNESTHYQILLTSFPHMENHSC
FEHMHHIPAPRPEEFHQRSNVTLTLRNLKGCCRHQVQIQPFFSSCLNDC
LRHSATVSCPEMPDTPEPIPDYMPLWVYWFITGISILLVGSVILLIVCM
TWRLAGPGSEKYSDDTKYTDGLPAADLIPPPLKPRKVWIIYSADHPLYV
DVVLKFAQFLLTACGTEVALDLLEEQAISEAGVMTWVGRQKQEMVESNS
KIIVLCSRGTRAKWQALLGRGAPVRLRCDHGKPVGDLFTAAMNMILPDF
KRPACFGTYVVCYFSEVSCDGDVPDLFGAAPRYPLMDRFEEVYFRIQDL
EMFQPGRMHRVGELSGDNYLRSPGGRQLRAALDRFRDWQVRCPDWFECE
NLYSADDQDAPSLDEEVFEEPLLPPGTGIVKRAPLVREPGSQACLAIDP
LVGEEGGAAVAKLEPHLQPRGQPAPQPLHTLVLAAEEGALVAAVEPGPL
ADGAAVRLALAGEGEACPLLGSPGAGRNSVLFLPVDPEDSPLGSSTPMA
SPDLLPEDVREHLEGLMLSLFEQSLSCQAQGGCSRPAMVLTDPHTPYEE
EQRQSVQSDQGYISRSSPQPPEGLTEMEEEEEEEQDPGKPALPLSPEDL
ESLRSLQRQLLFRQLQKNSGWDTMGSESEGPSA

The term “IL-17RE” refers to a protein known as interleukin 17 receptor E. Human IL-17RE has the amino acid sequence of (UniProt Q8NFR9):

(SEQ ID NO: 3)
MGSSRLAALLLPLLLIVIDLSDSAGIGFRHLPHWNTRCPLASHTDDSF
TGSSAYIPCRTWWALFSTKPWCVRVWHCSRCLCQHLLSGGSGLQRGLF
HLLVQKSKKSSTFKFYRRHKMPAPAQRKLLPRRHLSEKSHHISIPSPD
ISHKGLRSKRTQPSDPETWESLPRLDSQRHGGPEFSFDLLPEARAIRV
TISSGPEVSVRLCHQWALECEELSSPYDVQKIVSGGHTVELPYEFLLP
CLCIEASYLQEDTVRRKKCPFQSWPEAYGSDFWKSVHFTDYSQHTQMV
MALTLRCPLKLEAALCQRHDWHTLCKDLPNATARESDGWYVLEKVDLH
PQLCFKFSFGNSSHVECPHQTGSLTSWNVSMDTQAQQLILHFSSRMHA
TFSAAWSLPGLGQDTLVPPVYTVSQARGSSPVSLDLIIPFLRPGCCVL
VWRSDVQFAWKHLLCPDVSYRHLGLLILALLALLTLLGVVLALTCRRP
QSGPGPARPVLLLHAADSEAQRRLVGALAELLRAALGGGRDVIVDLWE
GRHVARVGPLPWLWAARTRVAREQGTVLLLWSGADLRPVSGPDPRAAP
LLALLHAAPRPLLLLAYFSRLCAKGDIPPPLRALPRYRLLRDLPRLLR
ALDARPFAEATSWGRLGARQRRQSRLELCSRLEREAARLADLG

Murine IL17RE has the amino acid sequence of (UniProt Q8BH06):

(SEQ ID NO: 6)
MGSPRLAALLLSLPLLLIGLAVSARVACPCLRSWTSHCLLAYRVDKRFA
GLQWGWFPLLVRKSKSPPKFEDYWRHRTPASFQRKLLGSPSLSEESHRI
SIPSSAISHRGQRTKRAQPSAAEGREHLPEAGSQKCGGPEFSFDLLPEV
QAVRVTIPAGPKASVRLCYQWALECEDLSSPFDTQKIVSGGHTVDLPYE
FLLPCMCIEASYLQEDTVRRKKCPFQSWPEAYGSDFWQSIRFTDYSQHN
QMVMALTLRCPLKLEASLCWRQDPLTPCETLPNATAQESEGWYILENVD
LHPQLCFKFSFENSSHVECPHQSGSLPSWTVSMDTQAQQLTLHFSSRTY
ATFSAAWSDPGLGPDTPMPPVYSISQTQGSVPVTLDLIIPFLRQENCIL
VWRSDVHFAWKHVLCPDVSHRHLGLLILALLALTALVGVVLVLLGRRLL
PGSGRTRPVLLLHAADSEAQRRLVGALAELLRTALGGGRDVIVDLWEGT
HVARIGPLPWLWAARERVAREQGTVLLLWNCAGPSTACSGDPQAASLRT
LLCAAPRPLLLAYFSRLCAKGDIPRPLRALPRYRLLRDLPRLLRALDAQ
PATLASSWSHLGAKRCLKNRLEQCHLLELEAAKDDYQGSTNSPCGFSCL

The term “antibody” as used herein refers to a protein comprising at least two heavy (H) chains and two light (L) chains inter-connected by disulfide bonds which interacts with an antigen. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

The phrase “antibody fragment”, as used herein, refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1I-VH-CH1) which, together with complementary light chain polypeptides, form a pair of antigen-binding sites (Zapata et al., (1995) Protein Eng. 8:1057-1062; and U.S. Pat. No. 5,641,870).

A “human antibody” or “human antibody fragment”, as used herein, includes antibodies and antibody fragments having variable regions in which both the framework and CDR regions are derived from sequences of human origin. Human antibodies can also be isolated from synthetic libraries or from transgenic mice (e.g. xenomouse) provided the respective system yield in antibodies having variable regions in which both the framework and CDR regions are equivalent to the sequences of human origin.

Furthermore, if the antibody contains a constant region, the constant region also is derived from such sequences. Human origin includes, e.g., human germline sequences, or mutated versions of human germline sequences or antibody containing consensus framework sequences derived from human framework sequences analysis, for example, as described in Knappik et al., (2000) J Mol Biol 296:57-86).

The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Lazikani et al., (1997) J. Mol. Bio. 273:927-948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mol. Biol. 273:927-948.

A “humanized antibody” or “humanized antibody fragment” is defined herein as an antibody molecule which has constant antibody regions derived from sequences of human origin and the variable antibody regions or parts thereof or only the CDRs are derived from another species. For example a humanized antibody can be CDR-grafted, wherein the CDRs of the variable domain are from a non-human origin, while one or more frameworks of the variable domain are of human origin and the constant domain (if any) is of human origin.

The term “chimeric antibody” or “chimeric antibody fragment” is defined herein as an antibody molecule which has constant antibody regions derived from, or corresponding to, sequences found in one species and variable antibody regions derived from another species. Preferably, the constant antibody regions are derived from, or corresponding to, sequences found in humans, and the variable antibody regions (e.g. VH, VL, CDR or FR regions) are derived from sequences found in a non-human animal, e.g. a mouse, rat, rabbit or hamster.

The term “isolated antibody” refers to an antibody or antibody fragment that is substantially free of other antibodies or antibody fragments having different antigenic specificities. Moreover, an isolated antibody or antibody fragment may be substantially free of other cellular material and/or chemicals. Thus, in some aspects, antibodies provided are isolated antibodies which have been separated from antibodies with a different specificity. An isolated antibody may be a monoclonal antibody. An isolated antibody may be a recombinant monoclonal antibody. An isolated antibody that specifically binds to an epitope, isoform or variant of a target may, however, have cross-reactivity to other related antigens, e.g., from other species (e.g., species homologs).

The term “recombinant antibody”, as used herein, includes all antibodies that are prepared, expressed, created or segregated by means not existing in nature. For example antibodies isolated from a host cell transformed to express the antibody, antibodies selected and isolated from a recombinant, combinatorial human antibody library, and antibodies prepared, expressed, created or isolated by any other means that involve splicing of all or a portion of a human immunoglobulin gene, sequences to other DNA sequences or antibodies isolated from an animal (e.g., a mouse) that is transgenic or transchromosomal for human immunoglobulin genes or a hybridoma prepared therefrom. Preferably, such recombinant antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. A recombinant antibody may be a monoclonal antibody. In an embodiment, the antibodies and antibody fragment disclosed herein are isolated from the Ylanthia® antibody library as disclosed in U.S. Ser. No. 13/321,564 or U.S. Ser. No. 13/299,367, which both herein are incorporated by reference.

The term “monoclonal antibody” as used herein refers to a preparation of antibody molecules of single molecular composition. A monoclonal antibody composition displays a unique binding site having a unique binding specificity and affinity for particular epitopes.

The terms “antagonist of IL-17C” and an “IL-17C antagonist”, are used interchangeably herein and refer to any molecule which inhibits the activity or function of IL 17C. The term “IL 17C antagonist” includes, but is not limited to, antibodies or antibody fragments specifically binding to IL-17C. Preferably, an IL-17C antagonist in the present disclosure is an antibody specific for human IL-17C. Such an antibody may be of any type, such as a murine, a rat, a chimeric, a humanized or a human antibody.

The term “antagonistic antibody specific for IL-17C” or “antagonistic antibodies specific for IL-17C” refers to antibodies or antibody fragments specifically binding to IL-17C. More preferably an IL-17C antagonist is an antibody or antibody fragment, such as a monoclonal antibody, specifically binding to IL-17C and blocks the binding of IL-17C to receptors of IL-17C, wherein the receptors of IL-17C include IL-17RE and IL-17RA. Such an antibody may be of any type, such as a murine, a rat, a chimeric, a humanized or a human antibody.

As used herein, the terms “binds specifically to”, “specifically binds to”, “is specific to/for” or “specifically recognizes” refer to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. For example, a standard ELISA assay can be carried out to determine specific binding. The scoring may be carried out by standard color development (e.g. secondary antibody with horseradish peroxide and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative can be more than 10-fold. Typically, determination of binding specificity is performed by using not a single reference antigen, but a set of about three to five unrelated antigens, such as milk powder, BSA, transferrin or the like.

“Percent identity” between a query amino acid sequence and a subject amino acid sequence is the “Identities” value, expressed as a percentage, thai is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query amino acid sequence may be described by an amino acid sequence identified in one or more claims herein.

The query sequence may be 100% identical to the subject sequence, or it may include up to a certain integer number of amino acid alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or earboxy-terminal positions of the query sequence or anywhere between those terminal positions, interspersed either individually among the amino acids in the query sequence or in one or more contiguous groups within the query sequence.

In one embodiment, the present disclosure is directed to an injection device comprising a stable anti-IL-17C antibody formulation described herein. For subcutaneous delivery, the formulation may be administered via a suitable device, such as (but not limited to) a syringe; an injection device (e.g. the INJECT-EASE™ and GENJECT™ device); an infusion pump (such as e.g. Accu-Chek™); an injector pen (such as the GENPEN™; or a needleless device (e.g. MEDDECTOR™ and BIOJECTOR™).

The pharmaceutical formulation of the pharmaceutically active anti-IL-17C antibody in accordance with the disclosure can be administered as subcutaneous injection, whereby the administration is repeated several times with time intervals of 1, 2, 3, or 4 weeks. In one embodiment the pharmaceutical formulation of the pharmaceutically active anti-IL-17C antibody is administered once every week or once every two weeks. The full volume of the injection fluid is in most cases administered within a time period of 1 to 10 minutes, preferably 2 to 6 minutes, and most preferably 1 to 3 minutes.

### Embodiments

In one embodiment the present disclosure provides a pharmaceutical formulation for an antigen binding protein comprising that antigen binding protein, a buffering agent and a stabilizer. More particularly, the present disclosure provides a pharmaceutical formulation for an antigen binding protein comprising about 100 to 250 mg/mL antigen binding protein, about 100 mM of a buffering agent providing a pH of about 5.0 to about 7.0 and about 100 mM of a stabilizer. In another aspect the antigen binding protein has a self-interaction propensity.

In one embodiment, the present disclosure provides a pharmaceutical formulation for an antigen binding protein comprising about 100 to 250 mg/mL, 125 to 200 mg/mL, 130 to 180 mg/mL, 140±10 mg/mL, 150±10 mg/mL, 160±10 mg/mL, 170±10 mg/mL, 140 mg/mL, 150 mg/mL, 160 mg/mL, 170 mg/mL of an antigen binding protein. In a further embodiment the pharmaceutical formulation comprises an antigen binding protein comprising 140 mg/mL antigen binding protein, about 100 mM of a buffering agent providing a pH of about 5.0 to about 7.0 and about 100 mM of a stabilizer. In a further embodiment the pharmaceutical formulation comprises an antigen binding protein comprising 150 mg/mL antigen binding protein, about 100 mM of a buffering agent providing a pH of about 5.0 to about 7.0 and about 100 mM of a stabilizer. In a further embodiment the pharmaceutical formulation comprises an antigen binding protein comprising 160 mg/mL antigen binding protein, about 100 mM of a buffering agent providing a pH of about 5.0 to about 7.0 and about 100 mM of a stabilizer.

In another embodiment, the present disclosure provides a pharmaceutical formulation for an antigen binding protein comprising about 100 to 250 mg/mL antigen binding protein; about 100 mM of a histidine buffer and about 100 mM of a stabilizer. In a further embodiment the histidine buffer concentration is 100 mM. In a further embodiment the histidine buffer is histidine hydrochloride. In another embodiment the pharmaceutical formulation has a pH of about 5.0 to about 7.0. In another aspect the pharmaceutical formulation has a pH of 6.0. In another embodiment the antigen binding protein has a self-interaction propensity. In another embodiment the stabilizer is an amino acid. In a further aspect the amino acid is arginine. In another embodiment the arginine concentration is about 100 mM. In a further embodiment the arginine concentration is 100 mM. In one embodiment the pharmaceutical formulation further comprises about 0.005 to 0.05% (w/v) of a nonionic surfactant. In another embodiment the nonionic surfactant is polysorbate 20 or polysorbate 80. In a further embodiment the polysorbate 20 or polysorbate 80 concentration is about 0.02% (w/v).

In another embodiment, the present disclosure provides a pharmaceutical formulation for an antigen binding protein comprising about 100 to 250 mg/mL antigen binding protein; about 100 mM of a histidine buffer and about 100 mM of arginine. In a further embodiment the histidine buffer concentration is 100 mM. In a further embodiment the histidine buffer is histidine hydrochloride. In a further embodiment the arginine concentration is 100 mM. In another embodiment the pharmaceutical formulation has a pH of about 5.0 to about 7.0. In another aspect the pharmaceutical formulation has a pH of 6.0. In another embodiment the antigen binding protein has a self-interaction propensity. In further embodiments the pharmaceutical formulation further comprises about 0.005 to 0.05% (w/v) of a nonionic surfactant. In another embodiment the nonionic surfactant is polysorbate 20 or polysorbate 80. In a further embodiment the polysorbate 20 or polysorbate 80 concentration is about 0.02% (w/v). In another embodiment the polysorbate 20 or polysorbate 80 concentration is 0.02% (w/v).

In one embodiment the present disclosure provides a pharmaceutical formulation for an antigen binding protein for subcutaneous or intramuscular administration.

In one embodiment the present disclosure provides a pharmaceutical formulation for an antigen binding protein, wherein the antigen binding protein is an antibody or antibody fragment thereof. In another embodiment the antibody is a monoclonal antibody or antibody fragment thereof. In another embodiment the monoclonal antibody or antibody fragment thereof has a propensity for self-interaction. In another embodiment the monoclonal antibody or antibody fragment thereof has a propensity for self-interaction characterized by a kD (diffusion interaction parameter) value of less than −5 mL/g, or less than −10 mL/g, or less than −15 mL/g, or less than −20 mL/g. In another embodiment the monoclonal antibody or antibody fragment thereof has a propensity for self-interaction characterized by a kD (diffusion interaction parameter) value of less than −5 mL/g, or less than −10 mL/g, or less than −15 mL/g, or less than −20 mL/g wherein the kD value was determined using the dynamic light scattering method as described in Connolly et al., 2012, Biophys. J. Vol. 103 or Menzen et al., 2014, J. Pharm. Sci., Vol. 103 in phosphate buffered saline. In another embodiment the monoclonal antibody or antibody fragment thereof has a propensity for self-interaction characterized by a kD (diffusion interaction parameter) value of about −23 mL/g. In another embodiment the monoclonal antibody or antibody fragment thereof has a propensity for self-interaction characterized by a kD (diffusion interaction parameter) value of −23 mL/g.

In another embodiment the monoclonal antibody or antibody fragment thereof has a propensity for self-interaction in phosphate buffered saline. In another embodiment the monoclonal antibody or antibody fragment thereof has a propensity for self-interaction characterized by a kD (diffusion interaction parameter) value of less than −5 mL/g, or less than −10 mL/g, or less than −15 mL/g, or less than −20 mL/g in phosphate buffered saline. In another embodiment the monoclonal antibody or antibody fragment thereof has a propensity for self-interaction characterized by a kD (diffusion interaction parameter) value of less than −5 mL/g, or less than −10 mL/g, or less than −15 mL/g, or less than −20 mL/g in phosphate buffered saline wherein the kD value was determined using the dynamic light scattering method as described in Connolly et al., 2012, Biophys. J. Vol. 103 or Menzen et al., 2014, J. Pharm. Sci., Vol. 103 in phosphate buffered saline. In another embodiment the monoclonal antibody or antibody fragment thereof has a propensity for self-interaction characterized by a kD (diffusion interaction parameter) value of about −23 mL/g in phosphate buffered saline. In another embodiment the monoclonal antibody or antibody fragment thereof has a propensity for self-interaction characterized by a kD (diffusion interaction parameter) value of −23 mL/g in phosphate buffered saline.

In another embodiment the antibody or antibody fragment thereof is an isolated antibody or antibody fragment thereof or a recombinant antibody or antibody fragment thereof. In another embodiment the monoclonal antibody or antibody fragment thereof is a human antibody or antibody fragment, a humanized antibody or antibody fragment or a chimeric antibody or antibody fragment.

In one embodiment, the present disclosure provides a pharmaceutical formulation for an antigen binding protein comprising about 100 to 250 mg/mL antigen binding protein; about 100 mM of a histidine buffer and about 100 mM of arginine, wherein the antigen binding protein is an IL-17C antibody or antibody fragment thereof. In a further embodiment the histidine buffer is histidine hydrochloride. In one embodiment the IL-17C antibody or antibody fragment thereof binds to human IL-17C (SEQ ID NO: 1). In another embodiment the IL-17C antibody or antibody fragment thereof comprises heavy and light chain variable regions comprising amino acid sequences that are 90% identical to SEQ ID NOs: 16 and 17 respectively. In another aspect the IL-17C antibody or antibody fragment thereof comprises heavy and light chain variable regions comprising amino acid sequences according to SEQ ID NOs: 16 and 17 respectively. In another embodiment the IL-17C antibody comprises heavy and light chains comprising amino acid sequences that are 90% identical to SEQ ID NOs: 18 and 19 respectively. In another aspect the IL-17C antibody comprises heavy and light chains comprising amino acid sequences according to SEQ ID NOs: 18 and 19 respectively. In a further embodiment the histidine buffer concentration is 100 mM. In a further embodiment the histidine buffer is histidine hydrochloride. In a further embodiment the arginine concentration is 100 mM. In another embodiment the pharmaceutical formulation has a pH of about 5.0 to about 7.0. In another aspect the pharmaceutical formulation has a pH of 6.0. In further embodiments the pharmaceutical formulation further comprises about 0.005 to 0.05% (w/v) of a nonionic surfactant. In another embodiment the nonionic surfactant is polysorbate 20 or polysorbate 80. In a further embodiment the polysorbate 20 or polysorbate 80 concentration is about 0.02% (w/v). In another embodiment the polysorbate 20 or polysorbate 80 concentration is 0.02% (w/v).

In one embodiment, the present disclosure provides a pharmaceutical formulation for an antigen binding protein comprising 100 to 250 mg/mL antigen binding protein; 100 mM of a histidine buffer and 100 mM of arginine, wherein the antigen binding protein is an IL-17C antibody or antibody fragment thereof. In one embodiment the IL-17C antibody or antibody fragment thereof binds to human IL-17C (SEQ ID NO: 1). In another embodiment the IL-17C antibody or antibody fragment thereof comprises heavy and light chain variable regions comprising amino acid sequences that are 90% identical to SEQ ID NOs: 16 and 17 respectively. In another aspect the IL-17C antibody or antibody fragment thereof comprises heavy and light chain variable regions comprising amino acid sequences according to SEQ ID NOs: 16 and 17 respectively. In another embodiment the IL-17C antibody comprises heavy and light chains comprising amino acid sequences that are 90% identical to SEQ ID NOs: 18 and 19 respectively. In another aspect the IL-17C antibody comprises heavy and light chains comprising amino acid sequences according to SEQ ID NOs: 18 and 19 respectively. In a further embodiment the histidine buffer concentration is 100 mM. In a further embodiment the histidine buffer is histidine hydrochloride. In a further embodiment the arginine concentration is 100 mM. In another embodiment the pharmaceutical formulation has a pH of about 5.0 to about 7.0. In another aspect the pharmaceutical formulation has a pH of 6.0. In further embodiments the pharmaceutical formulation further comprises about 0.005 to 0.05% (w/v) of a nonionic surfactant. In another embodiment the nonionic surfactant is polysorbate 20 or polysorbate 80. In a further embodiment the polysorbate 20 or polysorbate 80 concentration is about 0.02% (w/v). In another embodiment the polysorbate 20 or polysorbate 80 concentration is 0.02% (w/v).

In one embodiment, the present disclosure provides a pharmaceutical formulation for an IL-17C antibody or antibody fragment thereof, wherein the formulation comprises

a) about 100 to 250 mg/mL of the IL-17C antibody or antibody fragment thereof

b) about 100 mM of a histidine buffer providing a pH of about 5.0 to about 7.0

c) about 100 mM of arginine, and

d) about 0.005 to 0.05% (w/v) of a nonionic surfactant.

In one embodiment, the present disclosure provides a pharmaceutical formulation for an IL-17C antibody or antibody fragment thereof, wherein the formulation comprises

a) about 100 to 250 mg/mL of the IL-17C antibody or antibody fragment thereof

b) about 100 mM of a histidine buffer providing a pH of about 5.0 to about 7.0

c) about 100 mM of arginine, and

d) about 0.02% (w/v) of a nonionic surfactant.

In one embodiment the nonionic surfactant is polysorbate 20 or polysorbate 80.

In one embodiment, the present disclosure provides a pharmaceutical formulation for an IL-17C antibody or antibody fragment thereof, wherein the formulation comprises

a) 100 to 250 mg/mL of the IL-17C antibody or antibody fragment thereof

b) 100 mM of a histidine buffer providing a pH of 6.0±0.2

c) 100 mM of arginine, and

d) 0.005 to 0.05% (w/v) of a nonionic surfactant.

In one embodiment, the present disclosure provides a pharmaceutical formulation for an IL-17C antibody or antibody fragment thereof, wherein the formulation comprises

a) 100 to 250 mg/mL of the IL-17C antibody or antibody fragment thereof

b) 100 mM of a histidine buffer providing a pH of 6.0±0.2

c) 100 mM of arginine, and

d) 0.02% (w/v) of a nonionic surfactant.

In one embodiment, the present disclosure provides a pharmaceutical formulation for an IL-17C antibody or antibody fragment thereof, wherein the formulation comprises

a) 100 to 250 mg/mL of the IL-17C antibody or antibody fragment thereof

b) 100 mM of a histidine buffer providing a pH of 6.0±0.2

c) 100 mM of arginine, and

d) 0.02% (w/v) of polysorbate 20.

In one embodiment, the present disclosure provides a pharmaceutical formulation for an IL-17C antibody or antibody fragment thereof, wherein the formulation comprises

a) 100 to 250 mg/mL of the IL-17C antibody or antibody fragment thereof

b) 100 mM of a histidine hydrochloride buffer providing a pH of 6.0±0.2

c) 100 mM of arginine, and

d) 0.005 to 0.05% (w/v) of a nonionic surfactant.

In one embodiment, the present disclosure provides a pharmaceutical formulation for an IL-17C antibody or antibody fragment thereof, wherein the formulation comprises

a) 100 to 250 mg/mL of the IL-17C antibody or antibody fragment thereof

b) 100 mM of a histidine hydrochloride buffer providing a pH of 6.0±0.2

c) 100 mM of arginine, and

d) 0.02% (w/v) of a nonionic surfactant.

In one embodiment, the present disclosure provides a pharmaceutical formulation for an IL-17C antibody or antibody fragment thereof, wherein the formulation comprises

a) 100 to 250 mg/mL of the IL-17C antibody or antibody fragment thereof

b) 100 mM of a histidine hydrochloride buffer providing a pH of 6.0±0.2

c) 100 mM of arginine, and

d) 0.02% (w/v) of polysorbate 20.

In one embodiment the IL-17C antibody or antibody fragment thereof binds to human IL-17C (SEQ ID NO: 1). In another embodiment the IL-17C antibody or antibody fragment thereof comprises a HCDR1 region comprising amino acid sequence SEQ ID NO: 7, a HCDR2 region comprising amino acid sequence SEQ ID NO: 8, a HCDR3 region comprising amino acid sequence of SEQ ID NO: 9, a LCDR1 region comprising amino acid sequence SEQ ID NO: 13, a LCDR2 region comprising amino acid sequence of SEQ ID NO: 14 and a LCDR3 region comprising amino acid sequence SEQ ID NO: 15. In another embodiment the IL-17C antibody or antibody fragment thereof comprises a HCDR1 region of amino acid sequence SEQ ID NO: 7, a HCDR2 region of amino acid sequence SEQ ID NO: 8, a HCDR3 region of amino acid sequence of SEQ ID NO: 9, a LCDR1 region of amino acid sequence SEQ ID NO: 13, a LCDR2 region of amino acid sequence of SEQ ID NO: 14 and a LCDR3 region of amino acid sequence SEQ ID NO: 15. In another embodiment the IL-17C antibody or antibody fragment thereof comprises heavy and light chain variable regions comprising amino acid sequences that are 90% identical to SEQ ID NOs: 16 and 17 respectively. In another aspect the IL-17C antibody or antibody fragment thereof comprises heavy and light chain variable regions comprising amino acid sequences according to SEQ ID NOs: 16 and 17 respectively. In another embodiment the IL-17C antibody comprises heavy and light chains comprising amino acid sequences that are 90% identical to SEQ ID NOs: 18 and 19 respectively. In another aspect the IL-17C antibody comprises heavy and light chains comprising amino acid sequences according to SEQ ID NOs: 18 and 19 respectively.

In one embodiment, the present disclosure provides a pharmaceutical formulation for an IL-17C antibody, wherein the formulation comprises

a) 100 to 250 mg/mL of the IL-17C antibody

b) 100 mM of a histidine buffer providing a pH of 6.0±0.2

c) 100 mM of arginine,

d) 0.02% (w/v) of polysorbate 20,

and wherein the IL-17C antibody comprises heavy and light chain variable regions comprising amino acid sequences according to SEQ ID NOs: 16 and 17 respectively. In another embodiment the IL-17C antibody comprises heavy and light chains comprising amino acid sequences according to SEQ ID NOs: 18 and 19 respectively. In another embodiment the pharmaceutical formulation has a pH of 6.

In one embodiment, the present disclosure provides a pharmaceutical formulation for an IL 17C antibody, wherein the formulation comprises

a) 140 mg/mL of the IL-17C antibody

b) 100 mM of a histidine buffer providing a pH of 6.0±0.2

c) 100 mM of arginine,

d) 0.02% (w/v) of polysorbate 20,

and wherein the IL 17C antibody comprises heavy and light chain variable regions comprising amino acid sequences according to SEQ ID NOs: 16 and 17 respectively. In another embodiment the IL 17C antibody comprises heavy and light chains comprising amino acid sequences according to SEQ ID NOs: 18 and 19 respectively. In another embodiment the pharmaceutical formulation has a pH of 6.

In one embodiment, the present disclosure provides a pharmaceutical formulation for an IL 17C antibody, wherein the formulation comprises

a) 150 mg/mL of the IL-17C antibody

b) 100 mM of a histidine buffer providing a pH of 6.0±0.2

c) 100 mM of arginine,

d) 0.02% (w/v) of polysorbate 20,

and wherein the IL 17C antibody comprises heavy and light chain variable regions comprising amino acid sequences according to SEQ ID NOs: 16 and 17 respectively. In another embodiment the IL 17C antibody comprises heavy and light chains comprising amino acid sequences according to SEQ ID NOs: 18 and 19 respectively. In another embodiment the pharmaceutical formulation has a pH of 6.

In one embodiment, the present disclosure provides a pharmaceutical formulation for an IL-17C antibody, wherein the formulation comprises

a) 160 mg/mL of the IL-17C antibody

b) 100 mM of a histidine buffer providing a pH of 6.0±0.2

c) 100 mM of arginine,

d) 0.02% (w/v) of polysorbate 20,

and wherein the IL-17C antibody comprises heavy and light chain variable regions comprising amino acid sequences according to SEQ ID NOs: 16 and 17 respectively. In another embodiment the IL-17C antibody comprises heavy and light chains comprising amino acid sequences according to SEQ ID NOs: 18 and 19 respectively. In another embodiment the pharmaceutical formulation has a pH of 6.

In one embodiment, the present disclosure provides a pharmaceutical formulation for an IL-17C antibody, wherein the formulation comprises

a) 100 to 250 mg/mL of the IL-17C antibody

b) 100 mM of a histidine hydrochloride buffer providing a pH of 6.0±0.2

c) 100 mM of arginine,

d) 0.02% (w/v) of polysorbate 20,

and wherein the IL-17C antibody comprises heavy and light chain variable regions comprising amino acid sequences according to SEQ ID NOs: 16 and 17 respectively. In another embodiment the IL-17C antibody comprises heavy and light chains comprising amino acid sequences according to SEQ ID NOs: 18 and 19 respectively. In another embodiment the pharmaceutical formulation has a pH of 6.

In one embodiment, the present disclosure provides a pharmaceutical formulation for an IL 17C antibody, wherein the formulation comprises

a) 140 mg/mL of the IL-17C antibody

b) 100 mM of a histidine hydrochloride buffer providing a pH of 6.0±0.2

c) 100 mM of arginine,

d) 0.02% (w/v) of polysorbate 20,

and wherein the IL 17C antibody comprises heavy and light chain variable regions comprising amino acid sequences according to SEQ ID NOs: 16 and 17 respectively. In another embodiment the IL 17C antibody comprises heavy and light chains comprising amino acid sequences according to SEQ ID NOs: 18 and 19 respectively. In another embodiment the pharmaceutical formulation has a pH of 6.

In one embodiment, the present disclosure provides a pharmaceutical formulation for an IL 17C antibody, wherein the formulation comprises

a) 150 mg/mL of the IL-17C antibody

b) 100 mM of a histidine hydrochloride buffer providing a pH of 6.0±0.2

c) 100 mM of arginine,

d) 0.02% (w/v) of polysorbate 20,

and wherein the IL 17C antibody comprises heavy and light chain variable regions comprising amino acid sequences according to SEQ ID NOs: 16 and 17 respectively. In another embodiment the IL 17C antibody comprises heavy and light chains comprising amino acid sequences according to SEQ ID NOs: 18 and 19 respectively. In another embodiment the pharmaceutical formulation has a pH of 6.

In one embodiment, the present disclosure provides a pharmaceutical formulation for an IL-17C antibody, wherein the formulation comprises

a) 160 mg/mL of the IL-17C antibody

b) 100 mM of a histidine hydrochloride buffer providing a pH of 6.0±0.2

c) 100 mM of arginine,

d) 0.02% (w/v) of polysorbate 20,

and wherein the IL-17C antibody comprises heavy and light chain variable regions comprising amino acid sequences according to SEQ ID NOs: 16 and 17 respectively. In another embodiment the IL-17C antibody comprises heavy and light chains comprising amino acid sequences according to SEQ ID NOs: 18 and 19 respectively. In another embodiment the pharmaceutical formulation has a pH of 6.

In one embodiment, the present disclosure provides a pharmaceutical formulation for an IL-17C antibody, wherein the formulation consists of

a) 100 to 250 mg/mL of the IL-17C antibody

b) 100 mM of a histidine hydrochloride buffer providing a pH of 6.0±0.2

c) 100 mM of arginine,

d) 0.02% (w/v) of polysorbate 20,

and wherein the IL-17C antibody comprises heavy and light chain variable regions comprising amino acid sequences according to SEQ ID NOs: 16 and 17 respectively. In another embodiment the IL-17C antibody comprises heavy and light chains comprising amino acid sequences according to SEQ ID NOs: 18 and 19 respectively. In another embodiment the pharmaceutical formulation has a pH of 6.

In one embodiment, the present disclosure provides a pharmaceutical formulation for an IL 17C antibody, wherein the formulation consists of

a) 140 mg/mL of the IL-17C antibody

b) 100 mM of a histidine hydrochloride buffer providing a pH of 6.0±0.2

c) 100 mM of arginine,

d) 0.02% (w/v) of polysorbate 20,

and wherein the IL 17C antibody comprises heavy and light chain variable regions comprising amino acid sequences according to SEQ ID NOs: 16 and 17 respectively. In another embodiment the IL 17C antibody comprises heavy and light chains comprising amino acid sequences according to SEQ ID NOs: 18 and 19 respectively. In another embodiment the pharmaceutical formulation has a pH of 6.

In one embodiment, the present disclosure provides a pharmaceutical formulation for an IL 17C antibody, wherein the formulation consists of

a) 150 mg/mL of the IL-17C antibody

b) 100 mM of a histidine hydrochloride buffer providing a pH of 6.0±0.2

c) 100 mM of arginine,

d) 0.02% (w/v) of polysorbate 20,

and wherein the IL 17C antibody comprises heavy and light chain variable regions comprising amino acid sequences according to SEQ ID NOs: 16 and 17 respectively. In another embodiment the IL 17C antibody comprises heavy and light chains comprising amino acid sequences according to SEQ ID NOs: 18 and 19 respectively. In another embodiment the pharmaceutical formulation has a pH of 6.

In one embodiment, the present disclosure provides a pharmaceutical formulation for an IL-17C antibody, wherein the formulation consists of

a) 160 mg/mL of the IL-17C antibody

b) 100 mM of a histidine hydrochloride buffer providing a pH of 6.0±0.2

c) 100 mM of arginine,

d) 0.02% (w/v) of polysorbate 20,

and wherein the IL-17C antibody comprises heavy and light chain variable regions comprising amino acid sequences according to SEQ ID NOs: 16 and 17 respectively. In another embodiment the IL-17C antibody comprises heavy and light chains comprising amino acid sequences according to SEQ ID NOs: 18 and 19 respectively. In another embodiment the pharmaceutical formulation has a pH of 6.

In one embodiment the pharmaceutical formulation according to the present disclosure is stable upon freezing and thawing.

In one embodiment the pharmaceutical formulation according to the present disclosure is for subcutaneous or intramuscular administration.

In one embodiment the pharmaceutical formulation according to the present disclosure is in liquid or reconstituted form.

In one embodiment the present disclosure provides an injection device comprising a pharmaceutical formulation according to the present disclosure.

In another embodiment the present disclosure provides a pharmaceutical formulation as disclosed herein for use in the treatment of a disease or condition which is amenable to treatment with an IL-17C antibody or antibody fragment thereof in a subject comprising administering a formulation according the present disclosure in a subject in an amount effective to treat the disease or condition. In one embodiment the disease or condition is an inflammatory disease or disorder. In another embodiment disease or condition is rheumatoid arthritis, psoriasis, pulmonary inflammation, COPD and/or the treatment of atopic dermatitis (AD), including moderate-to-severe AD.

In another embodiment the present disclosure provides for a method of treating a disease or condition which is amenable to treatment with an IL-17C antibody or antibody fragment thereof in a subject comprising administering a formulation according the present disclosure in a subject in an amount effective to treat the disease or condition. In one embodiment the disease or condition is an inflammatory disease or disorder. In another embodiment disease or condition is rheumatoid arthritis, psoriasis, pulmonary inflammation, COPD and/or the treatment of atopic dermatitis (AD), including moderate-to-severe AD.

In another embodiment the present disclosure provides for a kit comprising one or more vials containing the formulation according to the present disclosure and instructions for subcutaneous administration of the formulation to a patient.

In another embodiment the present disclosure provides for an injection device comprising a stable pharmaceutical formulation as described herein. In another embodiment the present disclosure provides a kit comprising one or more vials containing a formulation as described herein and instructions for subcutaneous administration of the formulation to a patient. In another embodiment the kit further comprises an injection device for subcutaneous administration of the formulation to a patient.

In another embodiment the present disclosure provides for a pharmaceutical formulation according to the present disclosure for therapeutic use, such as the treatment of inflammatory disorders like e.g. rheumatoid arthritis, psoriasis, pulmonary inflammation, COPD and/or the treatment of atopic dermatitis (AD), including moderate-to-severe AD.

In another embodiment the present disclosure provides a method of treating a disease or condition which is amenable to treatment with an anti-IL-17C antibody or antibody fragment thereof in a subject comprising administering a formulation according to the present disclosure in a subject in an amount effective to treat said disease or condition. In another embodiment said disease or condition is an inflammatory disorder, like e.g. rheumatoid arthritis, psoriasis, pulmonary inflammation, COPD and/or the treatment of atopic dermatitis (AD), including moderate-to-severe AD.

TABLE 1
Antibody sequences

| Antibody | | SEQ ID NO: | [aa]/DNA |
|---|---|---|---|
| MAB#1 | HCDR1 (Kabat) | SEQ ID NO: 7 | DYAMH |
| | HCDR2 (Kabat) | SEQ ID NO: 8 | YIGGVGEGTQYAESVKG |
| | HCDR3 (Kabat) | SEQ ID NO: 9 | GFAIRYYGFDY |
| | HCDR1 (Chothia) | SEQ ID NO: 10 | GFTVSDY |
| | HCDR2 (Chothia) | SEQ ID NO: 11 | GGVGEG |
| | HCDR3 (Chothia) | SEQ ID NO: 12 | GFAIRYYGFDY |
| | LCDR1 (Kabat & | SEQ ID NO: 13 | SGDKLGDKYAY |
| | Chothia) | | |
| | LCDR2 (Kabat & | SEQ ID NO: 14 | QDSKRPS |
| | Chothia) | | |
| | LCDR3 (Kabat & | SEQ ID NO: 15 | QVFTFPLVTT |
| | Chothia) | | |
| | VH | SEQ ID NO: 16 | EVQLLESGGGLVQPGGSLRLSCAASGFTVSDYAMHWVRQAPGKGL |
| | | | EWVSYIGGVGEGTQYAESVKGRFTISRDNSKNTLYLQMNSLRAEDTA |
| | | | VYYCARGFAIRYYGFDYWGQGTLVTVSS |
| | VL | SEQ ID NO: 17 | SYELTQPPSVSVSPGQTASITCSGDKLGDKYAYWYQQKPGQSPVLVIY |
| | | | QDSKRPSGIPERFSGSNSGNTATLTISGTQAEDEADYYCQVFTFPLVT |
| | | | TVFGGGTKLTVLGQ |
| | Heavy chain | SEQ ID NO: 18 | EVQLLESGGGLVQPGGSLRLSCAASGFTVSDYAMHWVRQAPGKGL |
| | (IgG1) | | EWVSYIGGVGEGTQYAESVKGRFTISRDNSKNTLYLQMNSLRAEDTA |
| | | | VYYCARGFAIRYYGFDYWGQGTLVTVSSASTKGPSVFPLAPSSKSTSG |
| | | | GTAALGCLVKDYFPEPVTVSWNSGALTSGVHTFPAVLQSSGLYSLSSV |
| | | | VTVPSSSLGTQTYICNVNHKPSNTKVDKRVEPKSCDKTHTCPPCPAPE |
| | | | LLGGPSVFLFPPKPKDTLMISRTPEVTCVVVDVSHEDPEVKFNWYVD |
| | | | GVEVHNAKTKPREEQYNSTYRVVSVLTVLHQDWLNGKEYKCKVSNK |
| | | | ALPAPIEKTISKAKGQPREPQVYTLPPSREEMTKNQVSLTCLVKGFYPS |
| | | | DIAVEWESNGQPENNYKTTPPVLDSDGSFFLYSKLTVDKSRWQQGN |
| | | | VFSCSVMHEALHNHYTQKSLSLSPGK |
| | Light chain | SEQ ID NO: 19 | SYELTQPPSVSVSPGQTASITCSGDKLGDKYAYWYQQKPGQSPVLVIY |
| | | | QDSKRPSGIPERFSGSNSGNTATLTISGTQAEDEADYYCQVFTFPLVT |
| | | | TVFGGGTKLTVLGQPKAAPSVTLFPPSSEELQANKATLVCLISDFYPGA |
| | | | VTVAWKADSSPVKAGVETTTPSKQSNNKYAASSYLSLTPEQWKSHRS |
| | | | YSCQVTHEGSTVEKTVAPTECS |
| | VL (DNA) | SEQ ID NO: 20 | tcctacgagctgacccagcccccctccgtgtccgtgtctcctggccagaccgcctccatc |
| | | | acctgttccggcgacaagctgggcgataagtacgcctactggtatcagcagaagcccg |
| | | | gccagtcccccgtgctggtcatctaccaggactccaagcggccctccggcatccctgag |
| | | | cggttctccggctccaactccggcaacaccgccaccctgaccatctccggcacccaggc |
| | | | cgaggacgaggccgactactactgccaggtgttcaccttccccctggtcaccaccgtgtt |
| | | | cggcggaggcaccaagctgaccgtgctgggccag |
| | VH (DNA) | SEQ ID NO: 21 | gaggtgcagctgctggaatccggcggaggactggtgcagcctggcggctccctgagac |
| | | | tgtcttgcgccgcctccggcttcaccgtgtccgactacgctatgcactgggtccgacagg |
| | | | cccctggcaagggcctggaatgggtgtcctatatcggcggcgtgggcgagggcaccca |
| | | | gtacgctgagtctgtgaagggccggttcaccatctcccgggacaactccaagaacacc |
| | | | ctgtacctgcagatgaactccctgcgggccgaggacaccgccgtgtactactgtgccag |
| | | | aggcttcgccatccggtactacggcttcgactactggggccagggcaccctggtcaccg |
| | | | tgtctagc |
| | Light chain | SEQ ID NO: 22 | tcctacgagctgacccagcccccctccgtgtccgtgtctcctggccagaccgcctccatc |
| | (DNA) | | acctgttccggcgacaagctgggcgataagtacgcctactggtatcagcagaagcccg |
| | | | gccagtcccccgtgctggtcatctaccaggactccaagcggccctccggcatccctgag |
| | | | cggttctccggctccaactccggcaacaccgccaccctgaccatctccggcacccaggc |
| | | | cgaggacgaggccgactactactgccaggtgttcaccttccccctggtcaccaccgtgtt |
| | | | cggcggaggcaccaagctgaccgtgctgggccagcctaaggccgctccctccgtgacc |
| | | | ctgttccccccatcctccgaggaactgcaggccaacaaggccaccctggtctgcctgat |
| | | | ctccgacttctaccctggcgccgtgaccgtggcctggaaggccgacagctctcctgtga |
| | | | aggccggcgtggaaaccaccaccccctccaagcagtccaacaacaaatacgccgcct |
| | | | cctcctacctgtccctgacccccgagcagtggaagtcccaccggtcctacagctgccag |
| | | | gtcacacacgagggctccaccgtggaaaagaccgtggcccctaccgagtgctcc |
| | Heavy chain | SEQ ID NO: 23 | gaggtgcagctgctggaatccggcggaggactggtgcagcctggcggctccctgagac |
| | (IgG1, DNA) | | tgtcttgcgccgcctccggcttcaccgtgtccgactacgctatgcactgggtccgacagg |
| | | | cccctggcaagggcctggaatgggtgtcctatatcggcggcgtgggcgagggcaccca |
| | | | gtacgctgagtctgtgaagggccggttcaccatctcccgggacaactccaagaacacc |
| | | | ctgtacctgcagatgaactccctgcgggccgaggacaccgccgtgtactactgtgccag |
| | | | aggcttcgccatccggtactacggcttcgactactggggccagggcaccctggtcaccg |
| | | | tgtctagcgcctccaccaagggcccctccgtgttccctctggccccctccagcaagtcca |
| | | | cctctggcggcaccgctgccctgggctgcctggtcaaggactacttccccgagcccgtg |
| | | | accgtgtcctggaactctggcgccctgacctccggcgtgcacaccttccctgccgtgctg |
| | | | cagtcctccggcctgtactccctgtcctccgtcgtgaccgtgccctccagctctctgggca |
| | | | cccagacctacatctgcaacgtgaaccacaagccctccaacaccaaggtggacaagc |
| | | | gggtggaacccaagtcctgcgacaagacccacacctgtcccccctgccctgcccctga |
| | | | actgctgggcggaccttccgtgttcctgttccccccaaagcccaaggacaccctgatgat |
| | | | ctcccggacccccgaagtgacctgcgtggtggtggacgtgtcccacgaggaccctgaa |
| | | | gtgaagttcaattggtacgtggacggcgtggaagtgcacaacgccaagaccaagccc |
| | | | agagaggaacagtacaactccacctaccgggtggtgtccgtgctgaccgtgctgcacc |
| | | | aggactggctgaacggcaaagagtacaagtgcaaggtgtccaacaaggccctgcctg |
| | | | cccccatcgaaaagaccatctccaaggccaagggccagccccgcgagccccaggtgt |
| | | | acacactgccccctagccgggaagagatgaccaagaaccaggtgtccctgacctgtct |
| | | | ggtcaagggcttctacccctccgacattgccgtggaatgggagtccaacggccagccc |
| | | | gagaacaactacaagaccaccccccctgtgctggactccgacggctcattcttcctgta |
| | | | ctccaagctgaccgtggacaagtcccggtggcagcagggcaacgtgttctcctgctccg |
| | | | tgatgcacgaggccctgcacaaccactacacccagaagtccctgtccctgagccccgg |
| | | | caag |

### WORKING EXAMPLES

### Working Example 1

### Determination of Self-Interaction Propensity of an IL17C Antibody

Elevated viscosity of high concentration liquid formulations of monoclonal antibodies can be explained by intermolecular interactions (Binabaji et al., 2015, Pharm Res. Vol. 32). These interactions are called self-association or self-interaction. The protein concentration dependency of self-interaction processes can be detected by light scattering methods e.g. dynamic light scattering. This assay principle is well established in science (e.g. Connolly et al., 2012, Biophys. J. Vol. 103; Menzen et al., 2014, J. Pharm. Sci., Vol. 103) and leads to the identification of the mutual diffusion coefficient of a molecule. By fitting the mutual diffusion coefficient of a molecule to the antibody concentration by a linear equation, the parameter kD (diffusion interaction parameter) can be calculated. This parameter is related to the second virial coefficient and antibodies having a propensity for self-interaction have a kD value below 0.

For MAB#1 a kD of −23.6±0.9 mL/g was determined using the dynamic light scattering method as described above in phosphate buffered saline. This value indicates attractive interaction between the antibody molecules, so called antibody self-interaction.

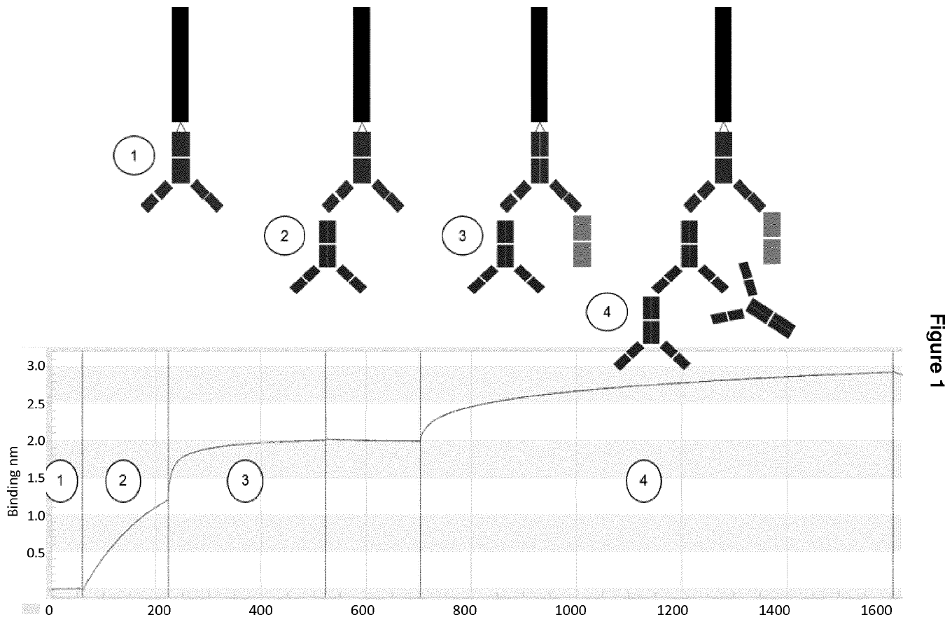

In order to find suitable formulations at high protein concentration for antibodies having a propensity to self-interact (such as MAB#1) a high-throughput method based on bio-layer-interferometry was established based on a publication by Sun et al. (2013, mAbs, Vol. 5). This method contains a capture step, where the antibody of interest is bound to a sensor surface (e.g. Anti-human Fc biosensor) by non-covalent interactions. Afterwards, a suitable saturation agent like human Fc protein saturates the sensor surface. The association reaction is observed for at least 1200 sec to make sure that equilibrium is established. A detailed description is depicted in FIG. 1.

A high signal intensity at equilibrium represents a high self-interaction propensity. As assay outcome, the parameter Rᵣₑₗ was introduced as ratio of the signal at association equilibrium and the amount of captured antibody. The results of this assay correlated with results obtained by dynamic light scattering methods and viscosity measurements (see Table 2).

TABLE 2
Correlation matrix of results obtained from DLS (k_{D}),
viscosimetry (increasing viscosity, k) and the
BLI-based self-interaction assay (Rᵣₑₗ). Significant spearman
correlations are indicated by p ≤ 0.05.

| | | Increasing | |
|---|---|---|---|
| Spearmans ρ | k_{D} | viscosity | Rᵣₑₗ |
| k_{D} | | −0.95 | −0.78 |
| k | −0.95 | | 0.77 |
| Rᵣₑₗ | −0.78 | 0.77 | |
| | | Increasing | |
| p-value | k_{D} | viscosity | Rᵣₑₗ |
| k_{D} | | 0.000 | 0.008 |
| k | 0.000 | | 0.009 |
| Rᵣₑₗ | 0.008 | 0.009 | |

### Working Example 2

### Identification of High Concentration Formulation for Antibodies Having a Self-Interaction Propensity

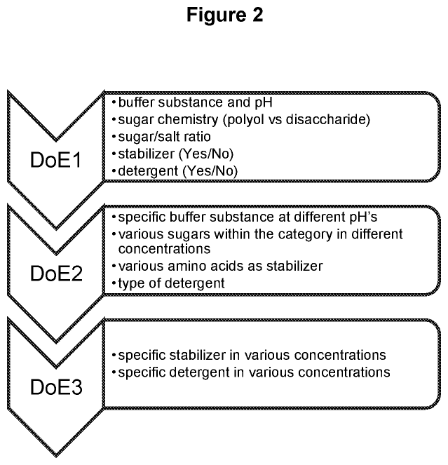

To find suitable formulation compositions a three-step screening approach was conceived based on Design of Experiments (DoE). Within this approach, several buffer substances like histidine hydrochloride, succinate hydrochloride, sodium phosphate or sodium citrate were tested in combination with excipients like sodium chloride, trehalose, sucrose, arginine, methionine, mannitol or sorbitol. A maximum theoretical osmolarity of 300 mOsm was chosen. Surfactants like Polysorbate 20 or Polysorbate 80 were also included in the study at several concentrations. An overview of the experimental design is shown in FIG. 2.

Each experimental design was tested in an accelerated stability study (2 weeks at 40° C., [Protein]=1 mg/mL). The assay setup consisted of high performance size-exclusion chromatography and the BLI-based self-interaction assay that was described above. By using the DoE software MODDE Pro (Umetrics™), the responses “Change in monomer portion” and “Self-interaction propensity” were optimized in each DoE setup. The goal was to calculate buffer compositions to minimize both, self-interaction propensity and change in monomer portion and to identify a high concentration formulation, which ideally is suitable for subcutaneous administration.

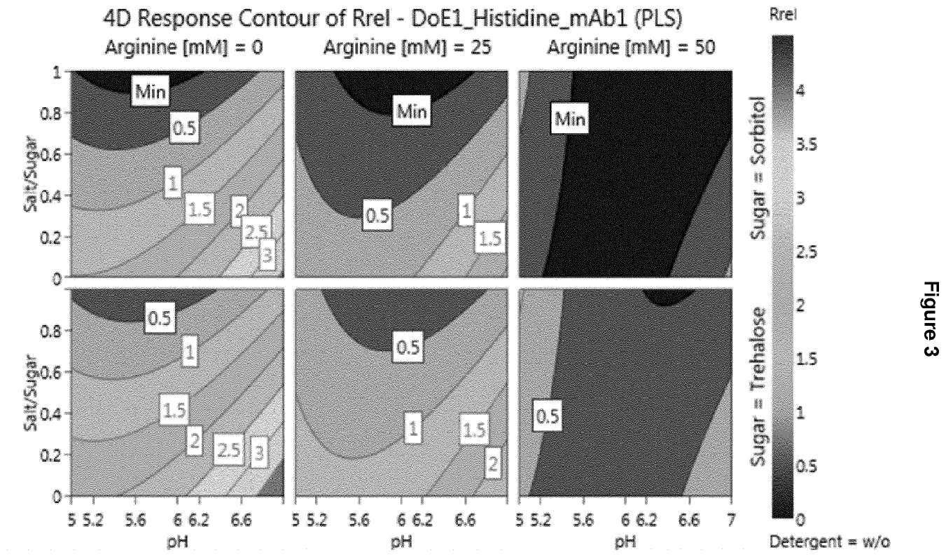

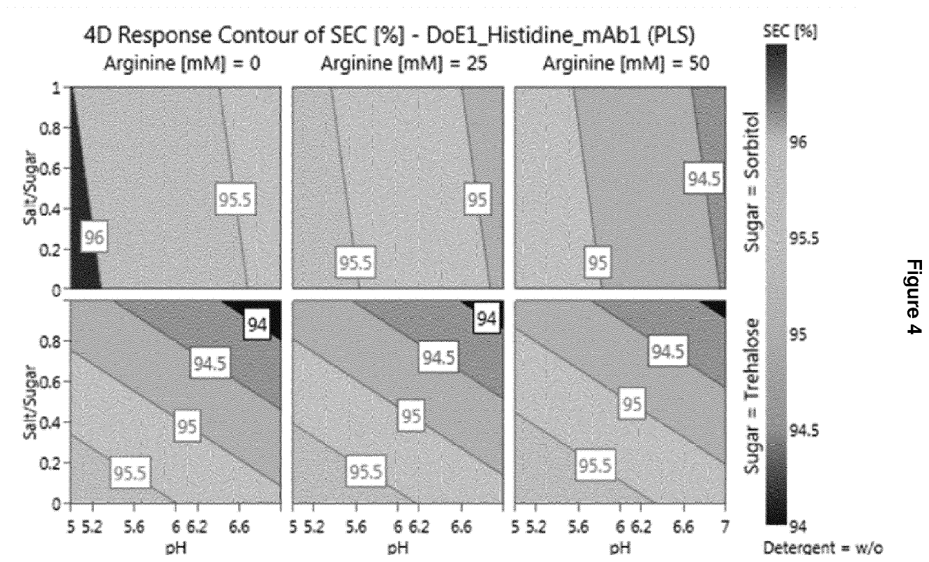

Within the first screening, several buffer substances at different pH-values, various salt/sugar ratios and the presence of stabilzer or surfactants were part of investigation. The outcome for the histidine-HCl based formulation is shown in FIG. 3 and FIG. 4.

By analyzing the monomer portion of MAB#1 (FIG. 4) the sugar chemistry together with the pH range showed the strongest influence on the antibody. A more acidic pH and sugar alcohols stabilized the antibody. Sorbitol in combination with arginine showed a destabilizing property whereas the combination of arginine and trehalose did not have any effects on the protein.

To minimize the self-interaction propensity of MAB#1 (FIG. 3), a high salt to sugar ratio, together with a high concentration of arginine seemed beneficial. The pH value was not critical if the above-mentioned criteria are met because of the predicted minimum of self-interaction in a pH range of 5.5 to 7. A pH value of 6.0 was defined as the best choice because of the highest buffer capacity according to the pK_{A} of 6.0. Additionally, the presence of detergent decreased the response given by the BLI-based assay. The reason for the curved lines in FIG. 3 was the existence of quadratic and interaction terms within the model. Therefore, other buffer combinations were also possible for minimizing self-interaction.

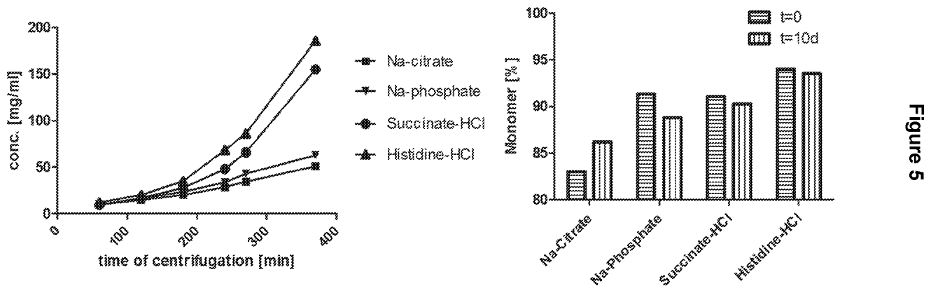

After the first screening round, an evaluation study was carried out. The antibody was formulated at low protein concentration in at least four different buffer composition containing either histidine hydrochloride, succinate hydrochloride, sodium phosphate or sodium citrate as buffer substance. The protein solution was then concentrated to at least 100 mg/mL and stored for 10 days at 5±3° C. The concentration procedure as well as size-exclusion chromatography analysis is shown in FIG. 5.

Histidine hydrochloride was chosen as the buffer substance for further investigation because the monomer stability was given (approx. 95% rel. Area in SEC experiments) and the viscosity was below 20 mPa*s at a protein concentration of 150 mg/mL. In addition, only limited effort was necessary to concentrate the protein in the chosen histidine hydrochloride formulation at lab scale. The following buffer was used for the next DoE-based screening round: 50 mM histidine-HCl pH 6.0, 50 mM arginine, 75 mM NaCl and 0.02% Tween 20.

In the second screening round (DoE2), the chosen formulation was further optimized in the by changing type and concentrations of excipients.

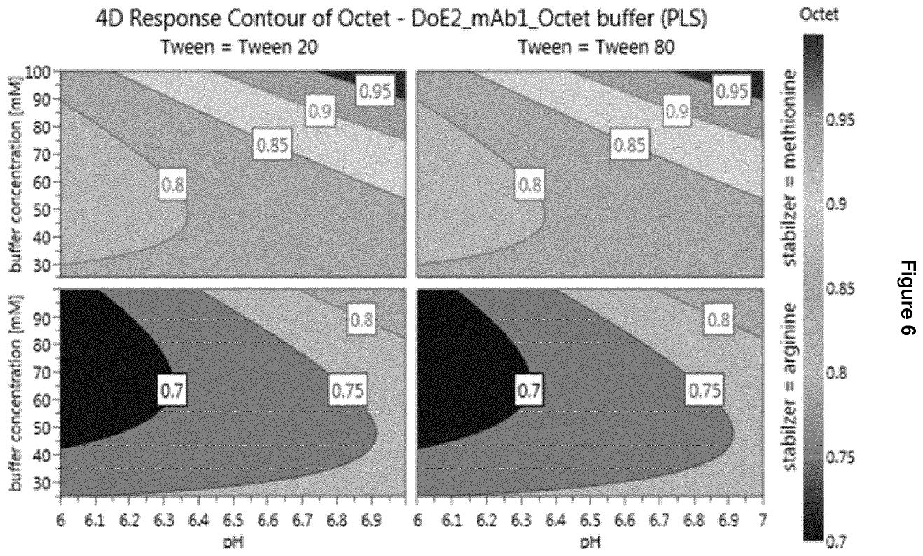

By screening for improved conditions to lower the self-interaction propensity of the protein, the presence of neither Tween 20 nor Tween 80 showed an important impact on the response (FIG. 6). However the presence of either methionine or arginine as stabilizer had an effect on the self-interaction. As depicted, a lower value of the response could be achieved by using 50 mM arginine instead of 50 mM methionine. That effect was independent of the buffer concentration or pH. For these two parameters, improved ranges could also be obtained. At high histidine concentrations with a pH value close to the pKa of 6.0, the lowest response of antibody self-interaction was observed.

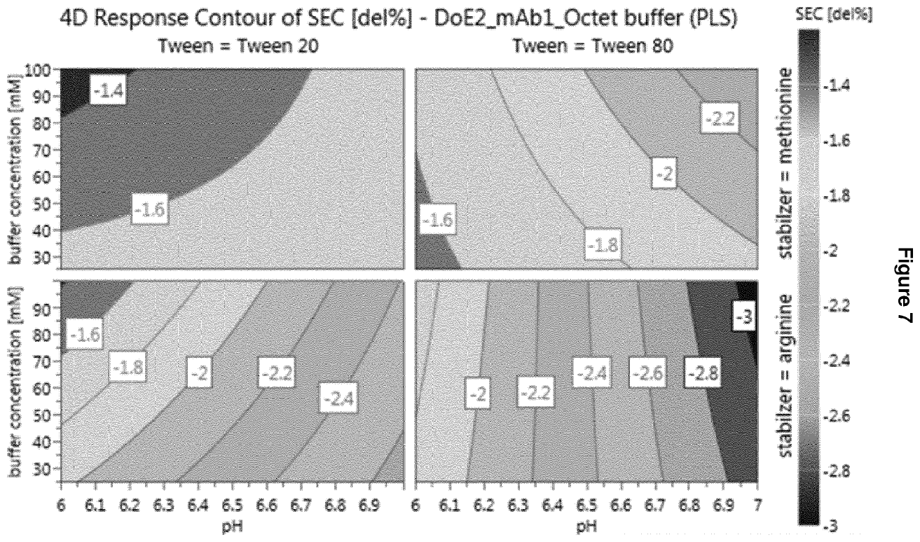

For the development of a formulation, the stabilizing effect to the protein regarding monomer portion is a very important criterion. It was therefore decided to choose a buffer system that lowers the self-interaction while maintaining a high monomer content as secondary criterion. FIG. 7 shows therefore the results of the formulation improvement regarding monomer content. The model suggested a destabilizing effect of Polysorbate 80 compared to Polysorbate 20. This impact could be weakened if methionine was present in the composition. This finding illustrated a stabilizing effect of methionine to the antibody monomer. As it was expected by the first screening round, a more acidic pH value maintained more monomer than a neutral pH value. As it was true for lowering self-interaction, a high concentration of histidine hydrochloride was preferred to maintain monomeric protein. The second screening round resulted in a formulation containing 100 mM histidine hydrochloride pH 6.0, 50 mM arginine and 0.02% Polysorbate 20. To fulfill the required theoretical osmolarity of approx. 300 mOsm, 25 mM sodium chloride were added.

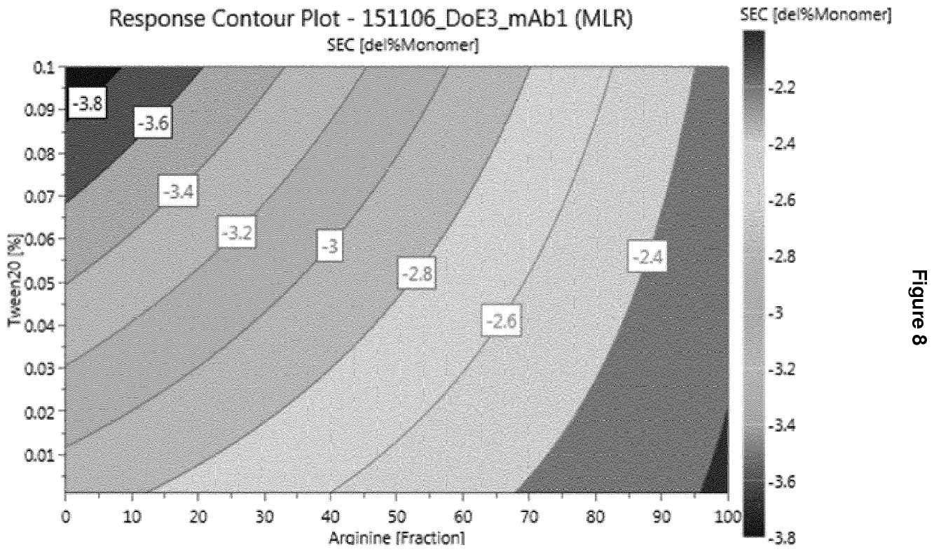

In the third screening reound (DoE3) the surfactant and stabilizer concentration was further optimized. DoE3 led to the conclusion that the highest possible concentration of arginine at low concentration of Polysorbate 20 resulted in minimal loss of monomer portion (FIG. 8). The response contour plot suggested, that the optimum of stabilization by arginine was not reached at 100 mM and that even higher concentrations might achieve more stability of the molecule. Interestingly, no valid model could be calculated for lowering the self-interaction propensity of MAB#1 because of the lack of significant effects. This result suggests that even the presence of arginine and detergents helps to decrease the antibody's tendency for self-association. Therefore, the low self-interaction propensity is robust within the tested ranges of arginine (<100 mM) and Polysorbate 20 concentrations (0.001-0.1% (v/v)).

The outcome of the third optimization was a formulation based on 100 mM histidine hydrochloride, 100 mM arginine and 0.001% (v/v) Polysorbate 20.

Because mechanical stress was not included in the DoE-based formulation development, a shaking stress study was initiated afterwards. The outcome of this study was that a higher polysorbate concentration is beneficial to maintain the monomer content of the antibody upon shaking (horizontal shaking, 400 rpm). The final MAB#1 high protein liquid formulation therefore has 160 mg/mL±10 mg/mL MAB#1 in 100 mM histidine hydrochloride, 100 mM arginine, 0.02% Polysorbate 20 at pH 6.0

### Working Example 3

### Stability Study and Shelf Life Assignment

A stability study was performed using scientifically sound analytical methods under non-GMP to assess the colloidal (UHP-SEC-MALS, CE-SDS) and chemical stability (HP-CEX). Moreover, the protein concentration was measured by UV-Vis spectroscopy and the concentration of active antibody content was analysed by SPR. For assuring suitability for subcutaneous administration, dynamic viscosity as parameter was included in this stability study. The samples are stored inverted at intended storage conditions (5° C.±3° C.) for up to 24 months and at accelerated storage conditions (25° C.±3° C.) for up to 6 months.

TABLE 3
Results for stability study of MAB#1 high protein liquid formulation, storage at 5° C. ± 3° C.
(unregulated humidity, inverted).

| | | Storage at 5° C. ± 3° C. | | | | | | |
|---|---|---|---|---|---|---|---|---|
| Analytical method/ | | 0 | 3 | 6 | 8 | 12 | 18 | 24 |
| Parameter | Unit | months | months | months | months | months | months | months |

Identity

| | | | | | | | | | |
|---|---|---|---|---|---|---|---|---|---|
| CEX- | Acidic | Area-% | 35 | 34 | 35 | 35 | 35 | 35 | 35 |
| HPLC | peak | | | | | | | | |
| | Main | | 55 | 56 | 55 | 54 | 54 | 51 | 53 |
| | peaks | | | | | | | | |
| | Basic | | 9 | 10 | 10 | 11 | 11 | 13 | 12 |
| | peaks | | | | | | | | |
| HIC- | Hydrophilic | Area-% | 0.5 | n.t. | n.t. | n.t | n.t | n.t. | 0.4 |
| HPLC | peak | | | | | | | | |
| | Main peak | | 99.5 | n.t. | n.t. | n.t | n.t. | n.t. | 99.7 |
| | Hydrophobic | | 0 | n.t. | n.t. | n.t. | n.t | n.t. | 0 |
| | peak | | | | | | | | |

Activity

| | | | | | | | | |
|---|---|---|---|---|---|---|---|---|
| hIL-17C active | % | 95 | 97 | 93 | 99 | 107 | 102 | 97 |
| concentration (rel. | | | | | | | | |
| activity) | | | | | | | | |

Purity and impurities

| Red. SDS-cGE | | % | 96.9 | 95.9 | 96.6 | 98.6 | 96.9 | n.t. | n.t. |
|---|---|---|---|---|---|---|---|---|---|
| Non-red. SDS-cGE | | % | 99.3 | 97.2 | 97.5 | 96.0 | 97.0 | n.t. | n.t. |
| Main peak | | | | | | | | | |
| Non-red. SDS-cGE | | % | 0.7 | 2.8 | 2.5 | 4.0 | 3.0 | n.t. | n.t. |
| Fragments | | | | | | | | | |
| UHP- | Monomer | Area-% | 96.9 | 96.6 | 96.3 | 96.3 | 96.1 | 95.5 | 95.6 |
| SEC | HMW | | 2.1 | 2.4 | 2.5 | 2.6 | 2.6 | 3.1 | 3.0 |
| | LMW | | 1.0 | 1.0 | 1.2 | 1.1 | 1.3 | 1.4 | 1.4 |

Viscosity

| | | | | | | | | |
|---|---|---|---|---|---|---|---|---|
| Viscosity | cP | 19.7 | n.t. | 21.0 | n.t. | n.t. | 19.9 | 21.0 |

12 months stability data show a slight tendency to reduced monomer (−0.8% over 12 months) accompanied by slight increase in aggregates (+0.5%) and fragments (+0.3%) over this time period in UHP-SEC, whereas no change in cGE profiles, CIEX charge pattern and binding activity could be observed over the 12 months (Table 3).

24 months stability data show a slight tendency to reduced monomer (−1.3% over 24 months) accompanied by slight increase in aggregates (+0.9%) and fragments (+0.4%) over this time period in UHP-SEC, whereas no change in cGE profiles, CIEX charge pattern and binding activity could be observed over the 24 months (Table 3).

These observations are confirmed by data derived from samples stored at accelerated conditions (25° C.±3° C.) where within 6 months the monomer content decreased by 2.9% accompanied by an increase of aggregates by 1.2% and fragments by 1.7% detected by UHP-SEC. The relative area of the main charge variant measured by CIEX dropped by 7% accompanied by increase of acidic species by 6% and increase of basic species by 2% .The relative binding activity of MAB#1 dropped by 9% compared to t0 (Table 4).

TABLE 4
Results for stability study of MAB#1 high protein liquid formulation, storage at 25° C. ± 3° C.
(unregulated humidity, upright), accelerated condition

| Analytical method/ | | | Storage at 25° C. ± 3° C. | | | |
|---|---|---|---|---|---|---|
| Parameter | | Unit | 0 months | 1 months | 3 months | 6 months |
| CEX-HPLC | Acidic peak | Area-% | 35 | 35 | 38 | 41 |
| | Main peaks | | 55 | 54 | 50 | 48 |
| | Basic peaks | | 9 | 12 | 12 | 11 |
| HIC-HPLC | Hydrophilic | Area-% | 0.5 | n.t. | n.t. | n.t |
| | peak | | | | | |
| | Main peak | | 99.5 | n.t. | n.t. | n.t. |
| | Hydrophobic | | 0 | n.t. | n.t. | n.t. |
| | peak | | | | | |

Activity

| | | | | | |
|---|---|---|---|---|---|
| hIL-17C active | % | 95 | 103 | 92 | 86 |
| concentration (rel. activity) | | | | | |

Purity and impurities

| Red. SDS-cGE | | % | 96.9 | 96.8 | 95.4 | 95.9 |
|---|---|---|---|---|---|---|
| Non-red. SDS-cGE | | % | 99.3 | 95.2 | 96.2 | 95.2 |
| Main peak | | | | | | |
| Non-red. SDS-cGE | | % | 0.7 | 4.8 | 3.8 | 4.9 |
| Fragments | | | | | | |
| UHP-SEC | Monomer | Area-% | 96.9 | 96.0 | 95.3 | 94.0 |
| | HMW | | 2.1 | 2.7 | 3.0 | 3.3 |
| | LMW | | 1.0 | 1.3 | 1.7 | 2.7 |

Viscosity

| | | | | | |
|---|---|---|---|---|---|
| Viscosity | cP | 19.7 | n.t. | n.t. | 21.3 |

Further, viscosimetry experiments were performed to determine the flow behavior of the MAB#1 formulation. The dynamic viscosity was measured on a Kinexus ultra plus cone-plate rheometer (Malvern) at a fixed temperature of 25° C. The rheometer was equipped with a measuring cone with an angle of 1° and a diameter of 40 mm. The experiment was performed with a measurement gap of 0.03 mm using ca. 80 μl of sample. A shear-ramp experiment was performed by using a shear-range of 10−1,000 s⁻¹. A constant dynamic viscosity below 20 mPa*s was observed across the tested shear-range, suggesting that the samples were similar to Newtonian systems.

1. A pharmaceutical formulation for an antigen binding protein comprising:
a) about 100 to 250 mg/mL antigen binding protein;
b) about 80 to 100 mM of a buffering agent providing a pH of about 5.0 to about 7.0; and
c) about 80 to 100 mM of a stabilizer.

2. A pharmaceutical formulation according to claim 1 wherein the buffering agent is a histidine buffer and wherein the stabilizer is an amino acid.

3. The pharmaceutical formulation according claim 2, wherein the histidine buffer concentration is about 100 mM.

4. The pharmaceutical formulation according to claim 3, wherein the histidine buffer concentration is 100 mM.

5. The pharmaceutical formulation according to any claim 2, wherein the histidine buffer is histidine hydrochloride and wherein the amino acid is arginine.

6. The pharmaceutical formulation according to claim 5, wherein the arginine concentration is about 100 mM.

7. The pharmaceutical formulation according to claim 6, wherein the arginine concentration is 100 mM.

8. The pharmaceutical formulation according to any claim 1, further comprising about 0.005 to 0.05% (w/v) of a nonionic surfactant.

9. The pharmaceutical formulation according to claim 8, wherein the nonionic surfactant is polysorbate 20 or polysorbate 80.

10. The pharmaceutical formulation according to claim 9, wherein the polysorbate 20 or polysorbate 80 concentration is 0.02% (w/v).

11. The pharmaceutical formulation according to claim 1, wherein the buffering agent provides a pH of 6.0±0.2.

12. The pharmaceutical formulation according to any claim 1 for subcutaneous or intramuscular administration.

13. The pharmaceutical formulation according to claim 1, wherein said antigen binding protein has a self-interaction propensity.

14. The pharmaceutical formulation according to claim 1, wherein said antigen binding protein is a monoclonal antibody or antibody fragment thereof.

15. The pharmaceutical formulation according to claim 14, wherein said monoclonal antibody or antibody fragment thereof specifically binds to human IL-17C (SEQ ID NO: 1).

16. The pharmaceutical formulation according to claim 15, wherein said monoclonal antibody or antibody fragment thereof comprises heavy and light chain variable regions comprising amino acid sequences that are 90% identical to SEQ ID NOs: 16 and 17 respectively.

17. The pharmaceutical formulation according to claim 16, wherein said monoclonal antibody or antibody fragment thereof comprises heavy and light chain variable regions comprising amino acid sequences according to SEQ ID NOs: 16 and 17, respectively.

18. An injection device comprising a pharmaceutical formulation according to claim 1.
